(19) United States
(12) Patent Application Publication
(10) Pub. No.: US 2025/0129330 A1
(43) Pub. Date: Apr. 24, 2025

(54) HYDROGEL-CARBON NANODOTS NANOCOMPOSITE FOR IN VITRO AND IN VIVO APPLICATIONS

(71) Applicant: UNIVERSITY OF OTTAWA, OTTAWA (CA)

(72) Inventors: DAVID J. LOMBONI, TORONTO (CA); RAFIK NACCACHE, LAVAL (CA); FABIO VARIOLA, OTTAWA (CA)

(21) Appl. No.: 18/924,222

(22) Filed: Oct. 23, 2024

Related U.S. Application Data
(60) Provisional application No. 63/592,749, filed on Oct. 24, 2023.
(60) Provisional application No. 63/595,585, filed on Nov. 2, 2023.

(51) Int. Cl. C12N 5/00

(52) U.S. Cl. CPC C12N 5/0068; C12N 2533/30; C12N 2533/54; C12N 2537/10; C12N 2539/00

(57) ABSTRACT

The present disclosure relates to injectable, aligneable and electroconductive hydrogel-carbon nanodots nanocomposite materials specifically designed to elicit key cellular functions relevant to in vitro, in vivo and clinical applications, such as neurogenic differentiation of eukaryotic stem cells and their electrophysiological maturation. The nanocomposites uniquely provide distinctive benefits attractive for both pre-clinical and clinical research, such as the potential to use various medically approved hydrogels, the ability to direct neurogenesis without exogenous factors, the capacity to support robust and directional axonal growth while eliciting the functional maturation of neurons through enhanced firing and a more active network activity, the potential for the in vitro and vivo assembly of 3D constructs of variable geometries to create specific architectures and/or follow specific anatomical trajectories, the facile integration of supplemental functions (e.g. drug release), ease of manufacturing and storage.

### FIELD

The present disclosure relates to injectable, aligneable and electroconductive hydrogel-carbon nanodots nanocomposite materials which elicit key functions in vitro and in vivo (such as, but not limited to, neurogenic differentiation and electrophysiological maturation) of eukaryotic multi- and pluri-potent stem cells as well as of bone, muscle, skin, fat, nerve, endothelial, sex, pancreatic and cancer cells.

### BACKGROUND

Pre-clinical Context That segment of in vitro research which employs stem cells and neurons (ranging from medicine and cellular biology to bioengineering) has greatly capitalized on the widely used two-dimensional (2D) monolayer cell culture systems, which rely on rigid plastic/glass culture plates and/or transwell inserts to support the adhesion and proliferation of cells. To this end, the bottom of conventional culture plates are usually functionalized with chemical groups (e.g. hydroxyl, carboxyl) and/or coated with proteins (e.g. poly-D-lysine, poly-L-lysine, collagen). These traditional assays are characterized by experimental simplicity and low cost, abundance of comparative literature and precise environmental control, all factors that have contributed to achieve an increasingly more sophisticated understanding of numerous cell-mediated processes.¹ Despite these advantages, recent research has shifted toward more complex 3D systems that recapitulates the hierarchical architecture and dynamic nature of native tissues.²⁻⁴

### Clinical Context

Neuronal disorders, including neurodegenerative conditions such as Alzheimer's and Parkinson's disease, as well as stroke and traumatic injuries (e.g. spinal cord injury), are associated with neuronal loss and/or the deterioration of neuroanatomical connectivity, severely affecting the quality of life of patients. The first attempts to regenerate lost neuronal functions date back to the 1970s and explored the transplantation of primary neurons sourced from human embryos.⁵⁻⁷ However, inconsistent protocols, low availability of fetal tissues and ethical concerns hindered their clinical success,⁸ inspiring researchers to turn to emerging alternatives for scalable and traceable sources, such as embryonic stem cells (ESCs) and induced pluripotent stem cells (iPSCs).

The well-established protocols and the efficacy in preclinical models have made stem cells an excellent candidate for cell-based therapies,⁵ but low (5-10%) survival rates, inadequate differentiation into the desired phenotype and adverse host immune responses, have negatively affected the therapeutic outcome.⁵⁻⁸ To address these limitations, researchers have turned to original approaches that synergistically integrate concepts of stem cell regenerative medicine with biomaterial-based tissue engineering.^{9,10} One of the fundamental design criteria for effective clinical translation in vivo emphasizes the use of biomaterials that (i) allow to effectively deliver cells in vivo and (ii) recapitulate key physicochemical features of the intended target tissue to recreate a biomimetic microenvironment that ultimately enhances cell survival, guides differentiation while promoting functional integration within the host tissue, among others.

For both pre-clinical and clinical applications, among the material properties deemed fundamental to support developmental processes associated with key stem cell and neural functions (e.g., cell proliferation and synaptic plasticity support), recent findings have highlighted the pivotal role of electroconductivity.⁹⁻¹⁴ Based on this evidence, the use of electroconductive hydrogels has emerged as an effective approach to recapitulate the physicochemical and electrical microenvironment of neural tissues both in vitro and in vivo.^{15,16} Notably, while widely used hydrogels (e.g., collagen, alginate, and gelatin) offer a cell-instructive microenvironment that mimics the physicochemical properties of native extracellular matrix (ECM) to enhance the adhesion, growth and differentiation of neuronal cell populations, they are characterized by a low electrical conductivity.¹⁷⁻¹⁹

Nanocomposites consisting of nanomaterials such as carbon nanotubes (CNTs) and graphene nanoparticles dispersed within a hydrogel matrix promote highly desirable cellular effects (e.g., neuronal differentiation, axonal elongation and network formation) and enhance electrical signaling among neurons as a result of the exceptional electrical conductivity of CNTs.²⁰⁻²² Likewise, nanomaterials including graphene oxide and reduced graphene oxide offer a conducive environment for cell adhesion and neurite expansion, in addition to modulating the expression of neuronal markers.²³⁻²⁵

### SUMMARY

The inventors have developed a novel electroconductive nanocomposite comprised of a collagen type-I matrix functionalized with carbon nanodots fabricated from glycine precursors (GlyCNDs). The rationale behind this novel strategy is that collagen has a fibrous structure that permit the biofunctionalization with nanoparticles and has already shown great promise as coating/matrix for in vitro cultures and as a therapeutic solution for central/peripheral nervous system injuries and degeneration.^{4,9,26} In parallel, the choice of CNDs, a relatively new type of carbon-based nanoparticles (1-10 nm in diameter), is supported by the fact that they offer diverse physicochemical properties and advantageous characteristics such as biocompatibility, low cytotoxicity, ease of synthesis, abundant functional groups (e.g., amino, hydroxyl, carboxyl) and high physicochemical stability.^{27,28}

In this disclosure, the inventors have synthesized and carried out a comprehensive physicochemical characterization of a collagen-GlyCND nanocomposite and evaluate its biological impact by employing both mouse induced pluripotent stem cells (iPSCs)-derived neural progenitor (NP) spheroids (and 3D cultures of primary cortical neurons as a confirmatory cell model). In particular, spheroids (hereafter also referred to as neurospheres) offer several advantages over conventional in vitro systems, making them a more physiologically relevant model for studying neural tissue engineering. Firstly, spheroids mimic the three-dimensional (3D) architecture of the ECM, thereby enabling more accurate cell-cell and cell-ECM interactions, which are essential to investigate cellular behavior and functions.²⁹ Secondly, spheroid cultures better recapitulate the spatial organization and cellular heterogeneity of native tissues, providing a more realistic environment for studying neuronal differentiation and network formation.^{29,30} Additionally, spheroids exhibit improved nutrient and oxygen gradients, closely resembling in vivo conditions.³¹ From a morphological point of view, significantly more neurite sprouting occurs in both spheroids and primary neurons when compared to collagen and collagen with suspended GlyCNDs, showing a significantly higher branching tendency. In addition, the GlyCNDs positively enhance additional functions, such as neuronal differentiation and electrophysiological maturation. In particular, the significantly lower number of cells positive for Ki-67, a nuclear proliferation marker, the lower expression of nestin (proliferating neuralprecursor marker) coupled with higher expression of both β-III-tubulin and MAP2 (early and mature neuronal marker), suggests that the nanocomposite accelerates neurodifferentiation of NP spheroids without exogenous factors. Moreover, the electrical activity displayed by the neural spheroids, as determined by multi-electrode arrays (MEA) measurements, was significantly higher when they were embedded in the nanocomposite (vs in collagen and in collagen with suspended GlyCNDs) in terms of both single electrode and network activity.

Thus, the present disclosure provides an injectable, aligneable and electroconductive hydrogel-nanodot nanocomposite material, comprising:
a physiologically acceptable polymer comprised of polymer chains/fibers with characteristic functional groups R″ along the polymer chains/fibers;
electrically conductive nanodots bearing preselected functional groups R′ decorating their outer surface; and
crosslinker agents having opposed ends and being bound at one end thereof to the functional groups R″ on the polymer chains/fibers and bound at the other end thereof to functional groups R′ on the outer surface of the electrically conductive nanodots, via covalent, electrostatic, or cathecol-based interactions.

The physiologically acceptable polymer is any one or combination of synthetic polymers and natural polymers.

The natural polymers may include any one or combination of collagen, agarose, gelatine, fibrin, elastin cellulose, silk fibroin, chitin, chitosan, glycosaminoglycans, keratin, pectin, hyaluronic acid, lactate, starch and lignin.

The synthetic polymers may include any one or combination of collagen, Polyethylene (PE), Polypropylene (PP), Polycarbonate (PC), Polyimide (PI), Polystyrene (PS), Poly(lactic-co-glycolic acid) (PLGA), Polylactic acid (PLA), Poly(lactic acid)-graft-poly(methacrylic acid) (PLA-g-P(MAA)), Poly-L-lysine (PLL), Polyethylene terephthalate (PET), Gelatin methacryloyl (GelMA), Polyethylene glycol diacrylate (PEGDA), Polyethylene glycol (PEG), Polyethylene oxide (PEO), Polyvinyl chloride (PVC), Polymethyl methacrylate (PMMA), Polytetrafluoroethylene (PTFE), Polycaprolactone (PCL), Polyvinyl alcohol (PVA), Polydimethylsiloxane (PDMS), Polyether ether ketone (PEEK), Polyhydroxyalkanoate (PHA)

The functional groups R″ may be any one or combination of NH₂, NH, NCO, CH₃, CH₂, COOH, CO, OH, SH, SO₃, H and O.

The electrically conductive nanodots may be any one or combination of carbon nanodots, graphene nanodots, metallic nanodots, semiconductor nanodots, silicon nanodots, indium tin oxide nanodots, copper nanodots and zinc oxide nanodots.

The functional groups R′ may be any one or combination of NH₂, COOH, NH, —OH, SH, O, SiOH, PO₃H₂, SO₃— and CONH₂.

The electrically conductive nanodots may have a diameter in a range from about 1 nm to about 100 nanometers.

The crosslinker molecules may be bound to the functional groups R′ and R″ by any one or combination of covalent bonding, electrostatic and cathecol-based interactions.

The crosslinker agents may be any one or combination of molecules selected on the basis of their ability for creating permanent covalent chemistries, dynamic covalent chemistries, ions capable of creating electrostatic interactions, and molecules for other classes of interactions.

The molecules selected on the basis of their ability for creating permanent covalent chemistries may include amine, amide, urea, thioether, siloxane and acrylamide bonds, the molecules selected on the basis of their ability for creating dynamic covalent chemistries may include imine, acylhydrazone, oxime,boronic-ester, disulfide and thioester bonds, ions selected on the basis of their ability for creating electrostatic interactions may include cation-anion, bridging ion, cation-π, anion-π, π-π interactions and hydrogen bonding, and the molecules for other classes of interactions may include cyclodextrin-based molecules, cathecol-based molecules and cucurbit[n]uril-based molecules.

The present disclosure provides a method of producing an injectable, aligneable and electroconductive hydrogel-nanodot nanocomposite material, comprising:
providing a physiologically acceptable polymer comprised of polymer chains/fibers forming a polymer matrix of the composite, functionalizing the polymer chains/fibers with preselected functional groups R″ attached along the polymer chains/fibers to produce functionalized polymer chains/fibers;
providing electrically conductive nanodots and functionalizing the electrically conductive nanodots with preselected functional groups R′ to an outer surface of the electrically conductive nanodots to producing functionalized electrically conductive nanodots; and
mixing the functionalized electrically conductive nanodots and the functionalized polymer strands with crosslinker molecules having opposed ends under conditions suitable induce formation of cross linkages between the electrically conductive nanodots and polymeric matrix through the crosslinker molecules having one end thereof bound to the R″ functional groups on the polymer strands and the other end of the crosslinker molecules bound to the R′ functional groups on the electrically conductive nanodots.
   The physiologically acceptable polymer is any one or combination of synthetic polymers and natural polymers.

The natural polymers may include any one or combination of collagen, agarose, gelatine, fibrin, elastin cellulose, silk fibroin, chitin, chitosan, glycosaminoglycans, keratin, pectin, hyaluronic acid, lactate, starch and lignin.

The synthetic polymers may include any one or combination of collagen, Polyethylene (PE), Polypropylene (PP), Polycarbonate (PC), Polyimide (PI), Polystyrene (PS), Poly(lactic-co-glycolic acid) (PLGA), Polylactic acid (PLA), Poly(lactic acid)-graft-poly(methacrylic acid) (PLA-g-P(MAA)), Poly-L-lysine (PLL), Polyethylene terephthalate (PET), Gelatin methacryloyl (GelMA), Polyethylene glycol diacrylate (PEGDA), Polyethylene glycol (PEG), Polyethylene oxide (PEO), Polyvinyl chloride (PVC), Polymethyl methacrylate (PMMA), Polytetrafluoroethylene (PTFE), Polycaprolactone (PCL), Polyvinyl alcohol (PVA), Polydimethylsiloxane (PDMS), Polyether ether ketone (PEEK), Polyhydroxyalkanoate (PHA)

The functional groups R″ may be any one or combination of NH₂, NH, NCO, CH₃, CH₂, COOH, CO, OH, SH, SO₃, H and O.

The electrically conductive nanodots may be any one or combination of carbon nanodots, graphene nanodots, metallic nanodots, semiconductor nanodots, silicon nanodots, indium tin oxide nanodots, copper nanodots and zinc oxide nanodots.

The functional groups R′ may be any one or combination of NH₂, COOH, NH, OH, SH, O, SiOH, PO₃H₂, SO₃— and CONH₂.

The electrically conductive nanodots may have a diameter in a range from about 1 nm to about 100 nanometers.

The crosslinker molecules may be bound to the functional groups R′ and R″ by any one or combination of covalent bonding, electrostatic and cathecol-based interactions.

The crosslinker agents may be any one or combination of molecules selected on the basis of their ability for creating permanent covalent chemistries, dynamic covalent chemistries, ions capable of creating electrostatic interactions, and molecules for other classes of interactions.

The molecules selected on the basis of their ability for creating permanent covalent chemistries may include amine, amide, urea, thioether, siloxane and acrylamide bonds, the molecules selected on the basis of their ability for creating dynamic covalent chemistries may include imine, acylhydrazone, oxime,boronic-ester, disulfide and thioester bonds, ions selected on the basis of their ability for creating electrostatic interactions may include cation-anion, bridging ion, cation-π, anion-π, π-π interactions and hydrogen bonding, and the molecules for other classes of interactions may include cyclodextrin-based molecules, cathecol-based molecules and cucurbit[n]uril-based molecules.

A further understanding of the functional and advantageous aspects of the invention can be realized by reference to the following detailed description and drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Embodiments will now be described, by way of example only, with reference to the drawings, in which:

FIGS. 1 to 10 show the physicochemical, electrical and structural stability characterization of the nanocomposite disclosed herein and relevant control conditions for comparison.

FIG. 1 is a plot of binding energy (eV) versus counts/s (a.u.) that shows the XPS survey scan of GlyCDs revealing five binding energies ascribed to Na1s, O1s, N1s and C1s.

FIG. 2 is a plot of Binding Energy (eV) versus Counts (a.u.) that shows representative high resolution XPS spectrum showing the deconvolution of the O1s region of GlyCNDs.

FIG. 3 is a plot of Wavelength (cm-1) versus Intensity (a.u.) that shows representative Raman spectra of GlyCNDs (insert), CE, C_CND1 and CE_CND1.

FIG. 4 is a plot of two vibrational C—N stretch vibrations versus normalized area (a.u.) that shows quantification of C—N stretching associated with the Amide III and Amide II groups, normalized against the N—C—H deformation of collagen proline ring.

FIG. 5 is a plot of wavelength (nm) versus fluorescent intensity (abs) that shows the fluorescence emission spectra at room temperature of GlyCNDs at excitation wavelengths of 320, 350 and 380 nm.

FIG. 6 is a plot of Time (days) versus GlyCNDs percent release that shows the release profile from collagen hydrogel of both pristine and EDC/NHS-activated GlyCNDs.

FIG. 7 shows an atomic force microscope (AFM) micrograph of CE_CND1 showing the spatial distribution of GlyCNDs within the collagen fibrous matrix.

FIG. 8 is a plot of Distance (nm) versus height (nm) that shows a representative depth profile extracted from AFM micrograph. Scale bar: 150 μm.

FIG. 9 is a plot of experimental conditions with increasing concentration of GlyCNDs versus Conductivity (mS/cm) that shows quantification of hydrogel conductivity with variable concentrations of GlyCNDs spanning from 0 (CE) to 4 mg/ml.

FIG. 10 is a plot of Time versus Mass loss that shows hydrolytic and enzymatic degradation of pristine Collagen (C), CE and CE_CND1. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 11 is a plot of experimental conditions versus LDH release (%) that shows LDH percentage release from different concentrations of GlyCND both immobilized within the collagen matrix (_) and suspended in culture medium (+). Pristine collagen (C) and EDC/NHS crosslinked collagen (CE) were employed as controls. Statistical significance was determined using one-way analysis of variance (ANOVA) and Tukey's Honestly Significant Difference (HSD) post hoc test: ** p<0.01, * p<0.05, non-significant (ns) p>0.05).

FIG. 12 is a representative PI/DAPI staining images that shows for control conditions C and CE, suspended GlyCNDs at a concentration of 1 mg/ml and 4 mg/ml and immobilized GlyCNDs at a concentration of 1 mg/ml and 4 mg/ml.

FIG. 13 is a plot of experimental conditions versus cell death (%) that shows cell death for the different conditions. Statistical significance was determined using one-way analysis of variance (ANOVA) and Tukey's Honestly Significant Difference (HSD) post hoc test: ** p<0.01, * p<0.05, non-significant (ns) p>0.05).

FIG. 14 is an image that shows the bioaccumulation (yellow arrow) of free-suspended GlyCNDs. Scale bar: 10 μm.

FIG. 15 is an image that shows representative 5 DIV brightfield images of spheroids embedded in CE, CE+rCND1 and CE_CND1 matrices. Scale bar: 200 μm.

FIG. 16 is a representative image that shows the β-III tubulin expression in neural networks in spheroids embedded in CE, CE_rCD1 and CE_CND1. Scale bar: 250 μm.

FIG. 17 is a representative SEM image that shows Semi-automated Sholl analysis performed on NP spheroids encompassing the use of 20 μm-spaced concentric hemispheres (centered around the spheroid's soma). Scale bar: 600 μm.

FIG. 18 is a plot of experimental conditions versus mean number of intersections/Schoenen index that shows number of neurite-hemisphere intersections and Schoenen ramification index. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 19 is a plot of distance (log) versus intersection/area (log) that shows a representative linear regression applied to the area-normalized intersections as a function of the distance from the spheroid's soma.

FIG. 20 is a plot of Sholl regression index versus experimental conditions that shows the Sholl's regression index. Statistical significance was determined using one-way analysis of variance (ANOVA) and Tukey's Honestly Significant Difference (HSD) post hoc test: ** p<0.01, * p<0.05, non-significant (ns) p>0.05).

FIG. 21 is a plot of experimental conditions versus neurite length (μm) that shows the quantification of neurites' length. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 22 is a plot of Relative GAP43 expression vs experimental conditions that shows the relative expression of GAP43. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 23 is an image that shows the 10-day expression of β III-tubulin and MAP2 in mouse primary cortical neurons cultured in CE, CE_CND1 and Matrigel. Nuclei were stained with DAPI. Scale bar: 100 μm.

FIG. 24 is representative immunofluorescence images that show the expression of Ki-67, MAP2 and β III-tubulin in spheroids cultured in CE, CE_rCND1 and CE_CND1 for 14 days. Nuclei were stained with DAPI. Scale bar: 100 μm.

FIG. 25 is a plot of experimental conditions versus Ki67 positive cells (%) that shows the quantification of the percentage of Ki-67 positive cells (Ki-67+). All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 26 is representative image that shows the Western blot analysis of β III-tubulin and MAP2 c/d expression.

FIG. 27 is an immunofluorescence image that shows the 14-day expression of nestin, calretinin and β III-tubulin in spheroids embedded in CE, CE+rCND1 and CE_CND1. Nuclei were stained with DAPI. Scale bar: 100 μm.

FIG. 28 is a plot of experimental conditions versus relative TUBB3 expression that shows the quantification of β III-tubulin expression levels. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 29 is a plot of experimental conditions versus relative MAP2 expression that shows the quantification of MAP2 expression levels. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 30 is representative image that shows a representative image of NP spheroids cultured in MEAs well plates. Scale bar: 300 μm.

FIG. 31 is a plot of time (sec) versus experimental conditions that shows activity traces at 14 days in vitro (DIV) for CE, CE_rCND1 and CE_CND1. Single detected spikes are represented by the black lines in the raster plots, while blue lines indicate single electrode burst. Neural networks are shown as pink rectangles.

FIG. 32 is a heatmap of time (days) versus experimental conditions vs mean firing rate (Hz) that shows the weighted mean firing rate during 28 days in vitro.

FIG. 33 is a plot of time (days in vitro) versus active electrodes that shows the number of detected active electrodes during 28 DIV. Shadowed areas represent mean±s.d.

FIG. 34 is a plot of average burst frequency (Hz) versus time (days in vitro) that shows the single electrode burst frequency. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 35 is a plot of time (days in vitro) versus network burst frequency (Hz) that shows the network burst frequency. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 36 is a plot of time (sec) versus electrodes that shows representative 1 s-binning raster plots showing singular spikes detected within network activity at 14 DIV for CE, CE_rCND1 and CE_CND1.

FIG. 37 is a plot of time (days in vitro) versus spikes per network that shows spikes per network activity during 28 DIV. Statistical significance was determined using one-way analysis of variance (ANOVA) and Tukey's Honestly Significant Difference (HSD) post hoc test: ** p<0.01, * p<0.05, non-significant (ns) p>0.05).

FIG. 38 is representative immunofluorescence images that show the 14-day expression of synapsin and β III-tubulin in spheroids cultured in CE, CE_rCND1 and CE_CND1 matrices. Nuclei were counterstained with DAPI. Scale bar: 100 μm.

FIG. 39 is representative image that shows Western blot analysis of synapsin and NMDAr expressions.

FIG. 40 is a plot of relative SYN expression versus experimental conditions that shows the quantification of synapsin expression levels. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 41 is representative immunofluorescence images that show NMDAr and β III-tubulin in spheroids cultured in CE, CE_rCND1 and CE_CND1 matrices. Nuclei were stained with DAPI. Scale bar: 100 μm.

FIG. 42 is a plot of relative NMDAr expression versus experimental conditions that shows the quantification of NMDAr expression levels. All numerical data are presented as mean±s.d. Statistical significance was determined using one-way analysis of variance (ANOVA) and Tukey's Honestly Significant Difference (HSD) post hoc test: ** p<0.01, * p<0.05, non-significant (ns) p>0.05).

FIG. 43 is a plot of time (days in vitro) versus firing rate change from baseline (%) that shows the percentage firing rate deviations from baseline recordings following acute MK-801 and NBQX treatment performed at 14 and 28 DIV. Statistical significance was determined using one-way analysis of variance (ANOVA) and Tukey's Honestly Significant Difference (HSD) post hoc test: ** p<0.01, * p<0.05, non-significant (ns) p>0.05).

FIG. 44 is a plot of time (days in vitro) versus burst frequency change from baseline (%) that shows the percentage firing rate deviations from baseline recordings following acute MK-801 and NBQX treatment performed at 14 and 28 DIV. Statistical significance was determined using one-way analysis of variance (ANOVA) and Tukey's Honestly Significant Difference (HSD) post hoc test: ** p<0.01, * p<0.05, non-significant (ns) p>0.05).

### DETAILED DESCRIPTION

Without limitation, the majority of the systems described herein are directed to a nanocomposite materials for eliciting cellular and tissue functions in vitro and in vivo, and method of using the same. As required, embodiments of the present invention are disclosed herein. However, the disclosed embodiments are merely exemplary, and it should be understood that the invention may be embodied in many various and alternative forms.

The accompanying figures, which are not necessarily drawn to scale, and which are incorporated into and form a part of the instant specification, illustrate several aspects and embodiments of the present disclosure and, together with the description therein, serve to explain the principles and some of the properties of the nanocomposite materials. The drawings are provided only for the purpose of illustrating select embodiments of the materials and as an aid to understanding and are not to be construed as a definition of the limits of the present disclosure. For purposes of teaching and not limitation, the illustrated embodiments are directed to nanocomposite materials consisting of a collagen matrix functionalized with carbon nanodots and method of using the same.

As used herein, the term “about”, when used in conjunction with ranges of dimensions, temperatures or other physical properties or characteristics is meant to cover slight variations that may exist in the upper and lower limits of the ranges of dimensions so as not to exclude embodiments where on average most of the dimensions are satisfied but where statistically dimensions may exist outside this region. For example, in embodiments of the present invention composition, physicochemical properties and biological characteristics of the nanocomposite materials may be given but it will be understood that these are not meant to be limiting.

As used herein, the terms “comprises”, “comprising”, “includes” and “including” are to be construed as being inclusive and open ended, and not exclusive. Specifically, when used in this specification including claims, the terms “comprises”, “comprising”, “includes” and “including” and variations thereof mean the specified features, steps or components are included. These terms are not to be interpreted to exclude the presence of other features, steps or components. As used herein, the term “injectable” means that the nanocomposite exhibits a gelation temperature of 37° C. and shear-thinning properties which allow it to be injected in the liquid form and successively form a solid hydrogel in the body, while preserving the viability of cells embedded therein during the injection process.

As used herein, the term “aligneable” means that the nanocomposite's structure consists of micro- and nano-metric fibers which can be aligned along a pre-determined direction.

As used herein, the phrase “physiologically acceptable polymer” means that the polymer does not trigger adverse immune reactions and/or toxic effects once injected in the body.

Neuronal disorders and traumas are characterized by the loss of functional neurons and disrupted neuroanatomical connectivity, severely impacting the quality of life of patients. This disclosure provides an electroconductive, aligneable nanocomposite consisting of glycine-derived carbon nanodots (GlyCNDs) incorporated into a collagen matrix and validates its beneficial physicochemical and electro-active cueing to relevant cells. To this end, the inventors employed mouse induced pluripotent stem cell (miPSC)-derived neural progenitor (NP) spheroids and 3D cultures of primary neurons. Findings revealed that the nanocomposite markedly augmented neuronal differentiation in NP spheroids, stimulated neuritogenesis and elicited early electrical activity.

In addition, the inventors have shown that the biomaterial-driven enhancements of the cellular response ultimately contributed to the development of highly integrated and functional neural networks. The inventors also propose a mechanism to demonstrate the establishment of direct interactions between collagen bound GlyCNDs and post-synaptic NMDA receptors based on the acute MK-801 treatment. In summary, the results establish a foundation for an innovative biomaterial-based tissue engineering approach aimed at addressing neuronal disorders by restoring damaged/lost neurons and re-establishing neuroanatomical connectivity. By integrating a carbon-based nanomaterial with a clinically approved hydrogel, this strategy offers a promising avenue for the development of more effective therapies targeting the underlying pathological substrate of neurological conditions.

The present disclosure will be illustrated using the following non-limiting example.

### Experimental Section

### Nanocomposite Hydrogels Preparation

While the present disclosure will be illustrated below using collagen, glycine-derived carbon nanodots and EDC-NHS coupling, it will be understood that additional synthetic and natural polymers, CNDs from different precursors and alternative bioconjugation protocols may be used. For example, additional biocompatible polymers (e.g. hydrogels for tissue engineering applications and bioinks), CNDs with different functional groups (e.g. —NH₂, —COOH, —NH, —OH) and variable surface distribution, as well as alternative protocols for covalent binding of the CNDs onto the polymeric matrix may be used. In addition, while the present disclosure will be illustrated below using mouse iPSC-derived neurospheres, it will be understood that additional eukaryotic stem/differentiated cells and their derived cellular constructs may be used. For example, monolayers and 3D constructs (e.g. scaffold-based 3D cultures, spheroids, neurospheres, organoids) of mouse/rat/human bone, muscle, skin, fat, nerve, endothelial, sex, pancreatic and cancer cells may be used.

Collagen hydrogels were prepared by dilution of rat tail type I stock (Corning, USA, 9.38 mg/ml, #354249) in differentiation medium (DM) and PBS 10× to reach a final concentration of 2 mg/ml. The medium recipe is outlined in “Derivation and culture of iPSCs-derived NP spheroids” section. GlyCNDs were introduced in the collagen solution either in their pristine or activated state. For the latter, GlyCNDs were immersed in MES buffer (pH=6.0) containing (1-Ethyl-3-[3-dimethylaminopropyl]carbodiimide hydrochloride) (EDC) and N-hydroxysuccinimide (NHS) (ratio 2:1) for 1 h at 4° C. The mass ratio between the GlyCNDs and EDC was fixed at 1:1. Upon the introduction of the GlyCNDs, the pH of the collagen solution was neutralized through the addition of 1 N NaOH. The collagen hydrogels were then incubated at 37° C. to allow complete polymerization. Three GlyCNDs concentrations were tested (for both pristine and activated GlyCNDs states) based on cytotoxic data available for other carbon-based nanoparticles in the literature (Table 1).³²⁻³⁴

TABLE 1
GlyCND concentrations tested in this study.
Pristine GlyCNDs

| Abbreviation | EDC/NHS [mM] | GlyCNDs [mg/mL] |
|---|---|---|
| C_CND05 | / | 0.5 |
| C_CND1 | / | 1.0 |
| C_CND4 | / | 4.0 |

Activated GlyCNDs

| | | |
|---|---|---|
| CE_CND05 | 2.5 | 0.5 |
| CE_CND1 | 5.0 | 1.0 |
| CE_CND4 | 20.0 | 4.0 |

### Atomic Force Microscopy (AFM)

The spatial distribution of the GlyCNDs within the collagen matrix was revealed by using non-contact AFM on am Alpha300 RSA system (WITec, Germany). Surfaces (1×1 μm²) were scanned using the triangular Si₃N₄ Cantilevers of the DNP-S10 chip (Bruker, USA), characterized by a nominal spring constant of 0.2 N m⁻¹, a resonant frequency of 13 kHz and nominal tip radius of 10 nm. 3D micrographs were then processed in Gwyddion74 to extract depth profile and GlyCNDs's diameter.

### XPS Characterization

GlyCNDs were analyzed using XPS with a K-Alpha X-ray photoelectron spectrometer (Thermo Scientific, USA). Three randomly selected regions of the sample were analyzed in triplicate, with each scan consisting of 10 runs. The results were averaged and plotted for both the survey and the high-resolution scans.

### Raman Spectroscopy

Single spectra for each experimental condition were acquired with the Raman module of the Alpha300 RSA system. Spectra were collected through a 50× objective (EC Epiplan NEOFLUAR, N.A.=0.9, Zeiss) with an excitation wavelength of 524 nm provided by a doubled Nd:YAG laser (12.5 mW, acquisition time of 2 s). Baseline subtraction, data normalization and Voigt (Lorentzian/Gaussian) deconvolution for the identification of vibrational components were performed in OriginPro (OriginLabs, USA). The bands related to C—N stretching of both the Amide III (1271 cm-1) and Amide II (1558 cm-1) groups were used to confirm the immobilization of the GlyCNDs into the collagen matrix.³⁵⁻³⁸ The 1098 cm⁻¹ band, associated to the N—C—H deformation of the collagen proline ring,^{36,37,39} was used as the reference peak for the normalization and comparison of spectra across conditions.

### Conductivity Measurements

A conductivity meter (CON 110 Conductivity/TDS Meters, Oakton® Instruments, USA) was used to assess the electrical conductivity of the collagen matrices used in this work, with a variable GlyCND concentration, namely 0.5, 1.0 and 4.0 mg/mL. The matrices were incubated at 37° C. for 2 h to allow for complete polymerization prior to the conductivity measurements. Three samples per concentration were analyzed and compared to the control condition represented by EDC-crosslinked collagen. Degradation studies: The structural stability of the collagen matrices with variable GlyCND concentration was evaluated by carrying out both hydrolytic and enzymatic degradation studies. For the latter, the degradation solution consisted of collagenase type IV from Clostridium histolyticum (305 U/mg, #LS004188, Worthington Biochemical Corporation, USA) dissolved in phosphate-buffered saline solution (PBS 1×) containing 2 mM CaCl₂) at a concentration of 10 U/ml. In both studies, 400 μl of degradation solution (PBS 1× for hydrolytic) was added to 250 μl of the matrix. At specific timepoints, the supernatant was collected, and the collagen concentration was estimated following the microplate procedure of the colorimetric BCA assay (Pierce™ BCA Protein Assay Kit, #23225, Thermo Fisher Scientific, USA) and calibration curves, obtained collagen standards (0-500 μg range) diluted in both degradation solutions. The percentage of the nanocomposites' mass loss was calculated at any selected timepoint by subtracting the collagen mass in the supernatant from the initial one (i.e., 500 μg).

Derivation and Culture of iPSCs-Derived NP Spheroids

Commercially available mouse iPSCs (Alstem, iPS02m) were propagated in feeder-free conditions on gelatin-coated culture surfaces. Cultures were periodically tested for mycoplasma with Lookout mycoplasma PCR detection kit (Sigma Aldrich, MP0035). iPSC maintenance medium was composed of KnockOut DMEM (Gibco, 10829018) supplemented with 15% knockout serum replacement (Gibco, N10828028), 1% MEM non-essential amino acid solution (Stemcell, 07600), 200 μM L-glutamine (Gibco, 25030), 1% penicillin-streptomycin (Gibco, 15070063), 100 μM 2-mercaptoethanol (Gibco, 31350) and 1000 U/ml leukemia inhibitory factor (LIF). Embryoid body (EB) formation was initiated by detaching iPSCs from culture surfaces using TryplE (Gibco, 12604013) and resuspending in fresh iPSC maintenance medium without LIF. Cell suspensions were transferred to Aggrewell 800 plates (Stemcell, 34811) treated with anti-adherence rinsing solution (Stemcell, 07010) and embryoid bodies were allowed to form overnight. Finally, EBs were transferred to anti-adherence treated 6 well plate. After 72 h, the media was switched to neuronal expansion (EM) consisting of 1:1 mixture of DMEM/F12 and Neurobasal medium (Gibco, #21103049) supplemented with 1% GlutaMAX, 1% pen/strep, 1% B-27™ Plus Supplement (Gibco, #A3582801), 0.5% N-2 Supplement (Gibco, #17502001), 200 μM ascorbic acid (Sigma Aldrich, #AX1775) and the following inhibitors: 5 μM SB-525334 (Tocris, 3211), 250 nM dorsomorphin (Tocris, 3093), 3 μM Wnt agonist CHIR99021 (Millipore, SML1046). After 10 days, the neurospheres expanded and formed spheroids. Prior to usage, the spheroids were passages 3× with TrypLE (ThermoFisher, #12604013). The resulting spheroids were embedded in the collagen matrices and culture for 4 days in differentiation medium (DM), consisting of EM without the inhibitors. The media was successively switched to maturation media (MM) consisting of DM supplemented with 100 μM brain-derived neurotrophic factor (BDNF, Stemcell Technologies, #78005), 100 μM glial derived neurotrophic factor (GDNF, Stemcell Technologies, #78058) and dybutyryl-cAMP (db-cAMP, Stemcell Technologies, #73882). During the spheroids culture, the maturation medium was refreshed every 3 days.

### Cytotoxicity Assays

To evaluate potential cytotoxic effects associated to both immobilized and medium-suspended GlyCNDs, the level of lactate dehydrogenase (LDH) was measured using CytoTox 96@Non-Radioactive (Promega, USA, G1780) assay. Briefly, 50 μl aliquots were collected from the culture media at 6 and 72 h and then transfer to a 96 well plate. Subsequently, 50 μl of the Citotox96 reagent were added to each sample aliquot. The plate was then covered with tin foil and incubated for 30 min at room temperature. Finally, 50 μl of the stop solution were added to each well and the absorbance band at 492 nm was collected using a Sinergy H1 plate reader (BioTech® Instruments, USA). The percent of cytotoxicity was calculated as:

$$Percent cytotoxicity = ( Experimental LDH release ( 0 D 490 ) / Maximum LDH release ( 0 D 490 ) ) \times 100$$

where the Maximum LDH release was obtained by adding 10 μL of 10× Lysis solution to a negative control sample, 1 h before adding the Cytotox96a Reagent. In addition, at 3 DIV, the samples were stained with PI (ThermoFisher, #BMS500PI) and Hoechst 33342 (ThermoFisher, R37605) and visualized under an LSM880 AxioObserverZ1 confocal microscope (Zeiss, Germany) with a Plan-Apochromat 20× objective (NA=0.8, Zeiss).

The resulting images were processed on ImageJ to quantify the number of PI-positive (PI+) nuclei that was used as a measure of cell death. According to the results obtained from the cytotoxicity assays, the 1 mg/ml concentration of GlyCNDs was selected (labelled CE_CND1) for all the subsequent cellular studies. This condition was compared with the control condition consisting of pristine collagen crosslinked with EDC/NHS (CE). Notably, we added an additional condition (labelled CE_CND1_s) in which the spheroids were embedded in a CE matrix, supplemented with GlyCNDs dispersed in the medium to simulate the release profile previously quantified for the E_CND1 condition. In this way, we were able to isolate the effect provided by both immobilized (CE_CND1) and dispersed unbound GlyCNDs released by the matrix (CE_CND1_s).

### Immunohistochemistry (IHC) Staining

At 14 DIV, the spheroids were fixed in fresh 4% paraformaldehyde (PFA) at room temperature for 2 h. Fixed samples were permeabilized with 0.25% Triton-X100 (Sigma-Aldrich, #11332481001) and blocked with 5% horse serum (ThermoFisher, #31874) overnight at 4° C. Samples were successively incubated with primary antibodies for 24 h at 4° C., rinsed for a minimum of 10 times with blocking buffer, and lastly incubated overnight at 4° C. with donkey secondary antibodies. The details and working dilutions of primary and secondary antibodies are listed in Table 2. After 5 rinses, the nuclei were stained with 4-6-diamidino-2-phenylindole-dihydrochloride (DAPI) for 4 h at room temperature.

TABLE 2
List of primary and secondary antibodies, with their
working dilution, used in this study.

| Primary antibody | Dilution | Secondary antibody | Dilution |
|---|---|---|---|
| β III-tubulin (Abcam, ab78078) | 1:1000 | anti-goat Alexa 488 | 1:500 |
| MAP2 (ThermoFisher, | 1:500 | anti-rat Alexa 555 | 1:500 |
| PA5-17646) | | anti-mouse Alexa 594 | 1:500 |
| Ki-67 (ThermoFisher, 14-569) | 1:250 | anti-rabbit Alexa 647 | 1:500 |
| Synapsin (Phosphosolution, | 1:500 | | |
| 1927-SYNP) | | | |
| NMDAR2D extracellular | 1:200 | | |
| (ThermoFisher, PA577425) | | | |
| Nestin (Abcam, ab105389) | 1:500 | | |
| Calretinin (Abcam, ab92341) | 1:500 | | |

### Protein Visualization

Spheroids were imaged on LSM880 AxioObserverZ1 confocal microscope through a Plan-Apochromat 20× objective (NA=0.8, Zeiss). The multi-channel z-stack images were successively processed in FIJI for background subtraction and the generation of a maximum projection.⁴⁰

### Morphological Analysis

Spheroids were imaged on an LSM880 AxioObserverZ1 confocal microscope (Zeiss, Germany) with a 10× EC Plan-Neofluar (Ph1) objective (NA=0.3, Zeiss). To evaluate neurite branching complexity and ramification, a semi-automated Sholl analysis centered around the spheroid body with a 20 μm-step between consecutive hemispheres was performed on ImageJ (Neurite Tracer plugin).⁴¹ The coefficient (Sholl's decay index) of the linear regression model was used as a measure of the rate of decay of the number of branches with distance from the center of analysis. As a measure of branch ramification, Schoenen index (i.e., maximum number of intersections divided by the number of primary branches) was used. In addition, a semi-automated Strahler analysis was performed to extract the mean branch length.

### Western Blotting

Spheroids cultured in CE, CE_rCND1 and CE_CND1 were harvested into cold RIPA buffer by scraping and sonication of the collagen matrix. The total protein concentration of the cell lysates was determined by BCA assay (Pierce™ BCA Protein Assay Kit, #23225, ThermoFisher). The lysates were successively boiled for 10 min at 95° C. in the sample loading buffer. The proteins were electrophoretically resolved on a 10% SDS-PAGE gel at 100 V. Resolved proteins were transferred to PVDF membranes for 30 min at 20 V using Transblot Turbo (BioRad, USA). After washing, the membranes were blocked in 5% BSA for 1 h at room temperature. Subsequently, the PVDF membrane were blotted with primary antibodies overnight at 4° C., washed 5 times with TBST buffer and incubated 2 h at room temperature with peroxidase-conjugated secondary antibodies. After washing, the membranes were imaged by a ChemiDoc XRS+ (BioRad) system and the bands were analyzed with the ImageJ software. Primary antibodies targeting the following proteins were used: β III-tubulin (ab78078), MAP2 (PA5-17646), synapsin (1927-SYNP), GAP43 (ab16053), NMDAR2D (PA577425).

### Multi-Electrode Arrays (MEA) Readings

The spheroids were cultured on Cytoview MEA 48-well plates (Axion BioSystem, USA, M768-tMEA-48B-5), after pre-coating with poly-Llysine and laminin. Baseline recording of spontaneous activity were performed in a Maestro MEA system and AxIS software (Axion Biosystems) by using a bandwidth with a filter for 10 Hz to 2.5 kHz cut-off frequencies. Spikes were detected by using an adaptive threshold set to 6 times the standard deviation of the estimated noise on each electrode. Each plate rested for 1 min for acclimatization in the Maestro instrument and was then recorded for an additional 2 min. Multi-electrode data analysis was performed using the Axion Biosystems Neural Metrics Tool. Bursts were identified in the data recorded from each individual electrode using an adaptive Poisson surprise algorithm. Network bursts were identified for each well using a nonadaptive algorithm requiring a minimum of 40 spikes and of 25% of active electrodes with a maximum inter-spike interval of 100 ms.

Based on baseline recording at 14 DIV, the most active wells electrodes (>0.1 spikes/sec) were selected for treatment with either dizocilpine (MK-801 maleate) or 2,3-dihydroxy-6-nitro-7-sulfamoyl-benzo(f)quinoxaline (NBQX). Briefly, the medium of the selected wells was complemented with either 0.1 M MQ-801 or 0.1 M NBQX. After 30 minutes of incubation, spontaneous neuronal activity was measured for 2 min to identify the effect of the specific blocker. After recordings, the samples were washed 5× with PBS 1× and fresh medium was added. The same procedure was followed for the treatment at 28 DIV. For each experimental condition, a total of 3 independent experiments were performed using a minimum of 9 wells per plate.

### Culture and Imaging of Mouse Primary Cortical Neurons

Primary cortical neurons from QBM Cell Science (Canada, #171003) were suspended in Neurobasal medium complemented with 1% GlutaMAX, 1% pen/strep, 1% B-27™ Plus Supplement, and 0.5% N-2 Supplement. To incorporate the cells into the hydrogels, the cell suspension and hydrogel solution were mixed in a 1:1 ratio, achieving a final cell density of 5.0×106 cells/ml and a final collagen concentration of 2 mg/ml. The cell-hydrogel mixture was then pipetted into glass bottom 96-well plates (Greiner Bio-One, #655892, USA) and incubated at 37° C. for 30 minutes to allow gelation. Successively, 200 μl of complete culture medium was added to each well. The hydrogels were maintained in culture for up to 7 days with media changes performed every 2 days. At 7 DIV, the samples were fixed in fresh 4% PFA at room temperature for 2 h. Fixed samples were permeabilized with 0.25% Triton-X100 and blocked with 5% horse serum (ThermoFisher, #31874) overnight at 4° C. Finally, the samples were stained for β III-tubulin and MAP2 and imaged on LSM880 AxioObserverZ1 confocal microscope through a Plan-Apochromat 20× objective (NA=0.8, Zeiss). The multi-channel z-stack images were successively processed in FIJI for background subtraction and the generation of a maximum projection.

### Data Analysis

Data are reported as mean±standard deviation (SD) or standard error of the mean (SEM) from at least 3 separate experiments. Data were plotted with GraphPad software, version 8.0. The normality of the distribution was assayed by different tests, such as Pearson normality test and Shapiro-Wilk normality test. For normally distributed data, one-way, two-way analysis of variance (ANOVA) test followed by Tukey's Honestly Significant Difference (HSD) post hoc test was used. For non-normally distributed data, Kolmogorov-Smirnov test analyses was carried out. Significance was set at p 0.05.

### Results and Discussion

### Characterization of the Nanocomposite

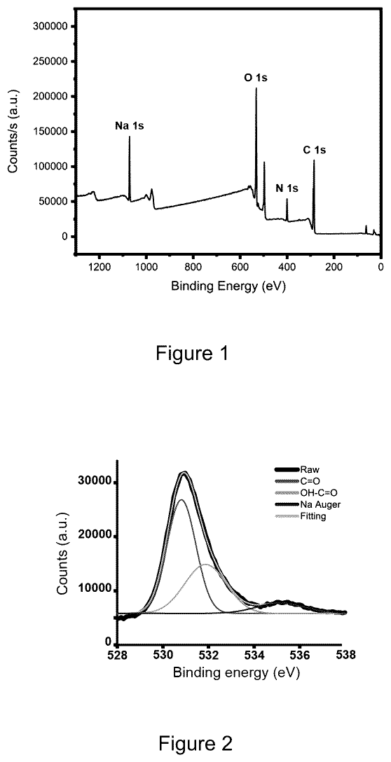

The detailed fabrication process and physicochemical characterization of the GlyCNDs have been previously reported by Naccache and co-authors.⁴² Here, we carried out a complementary high-resolution X-ray photoelectron spectroscopy (XPS) analysis (FIG. 1) to confirm the nature of the surface functional sites on the GlyCNDs. Upon deconvolution of the O1s peak, see FIG. 2, the inventors observed a substantial presence of COOH functional groups (531.87 eV), expected to create amide bonds as a result of their interactions with the free amines (NH2) along the collagen chain. 43 To test the resulting binding affinity, we incorporated the GlyCNDs in a collagen suspension at a concentration of 1 mg/ml both in their pristine state (hereafter referred to as C_CND1) and upon EDC/NHS activation (hereafter referred to as CE_CND1), and compared them to collagen crosslinked with EDC/NHS. The degree of immobilization was evaluated by Raman spectroscopy.

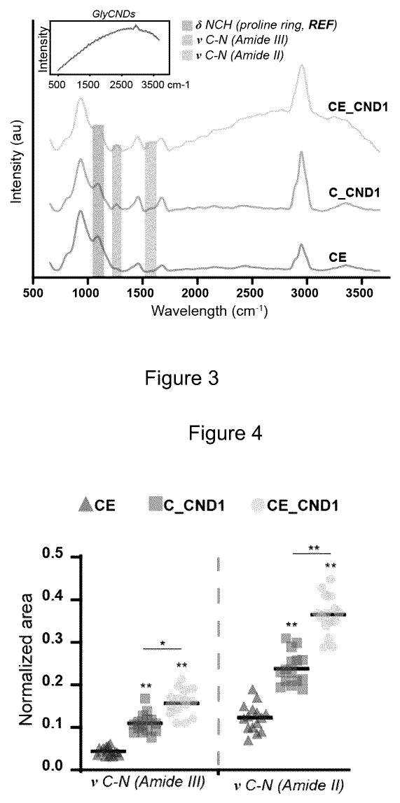

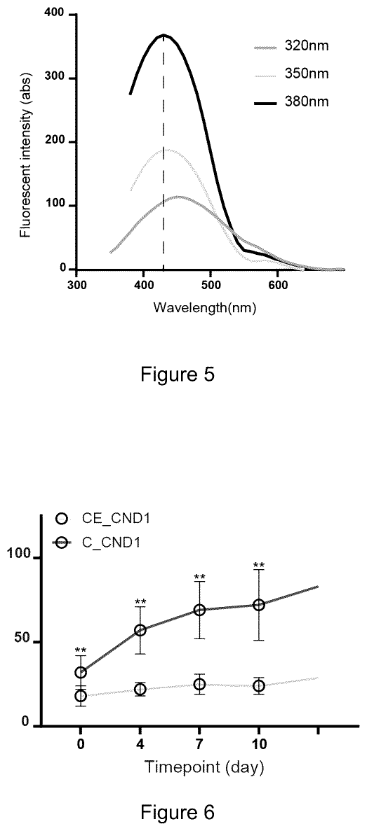

FIG. 3 displays the representative Raman spectra for CE, C_CND1 and CE_CND1. A greater presence of GlyCNDs in CE_CND1 is attested by the appearance of a wide fluorescence band spanning from ˜1800 to 3600 cm-1 that is characteristic of the nanoparticles (see Raman spectra of GlyCNDs in FIG. 3—insert). Furthermore, the formation of new C—N bonds between the COOH groups of GlyCNDs and NH2 groups of collagen is demonstrated by quantifying the area of the bands associated with the C—N stretching of both Amide III (1286 cm⁻¹) and II (1589 cm⁻¹).³⁵⁻³⁸ Specifically, the average normalized area relative to C—N stretching of Amide III (FIG. 4) is 0.04 for CE, and it increases to 0.12 and 0.17 for C_CND1 and CE_CND1, respectively. Finally, the normalized area of the C—N stretching of Amide II (FIG. 4) increases from 0.12 for CE to 0.24 and 0.38 for C_CND1 and CE_CND1, respectively. By exploiting the native fluorescence of GlyCNDs at an excitation wavelength of 380 nm (FIG. 5), we carried out release studies to further confirm the role of EDC/NHS coupling in achieving a stable immobilization of the GlyCNDs within the collagen matrix.

As shown in FIG. 6, the release of GlyCNDs displayed by CE_CND1 is significantly lower compared to that of C_CND1 condition throughout the full experiment timeframe (30 days). Notably, the release percentage from CE_CND1 at day 4 (˜20%) is used to include one additional control for the cellular experiments (referred to as CE+rCND1, and further described in Section 2.3), where the inventors supplemented the culture medium with the same amount of GlyCNDs released from the CE_CND1 condition at this time point. This enabled the inventors to isolate the cellular effects solely due to the immobilized GlyCNDs from those associated with suspended nanoparticles.

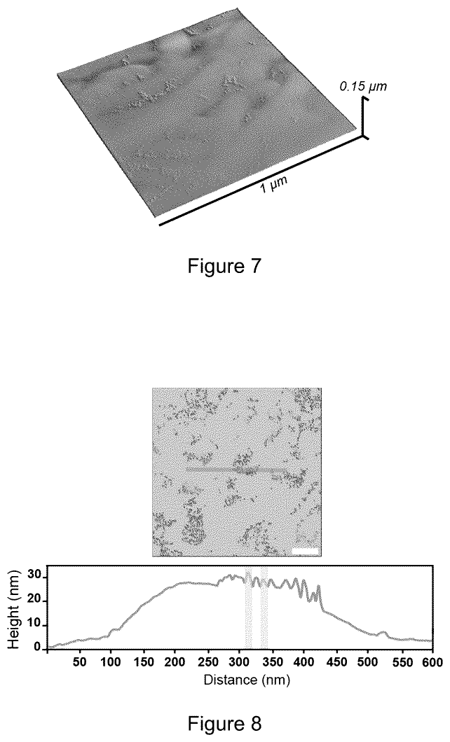

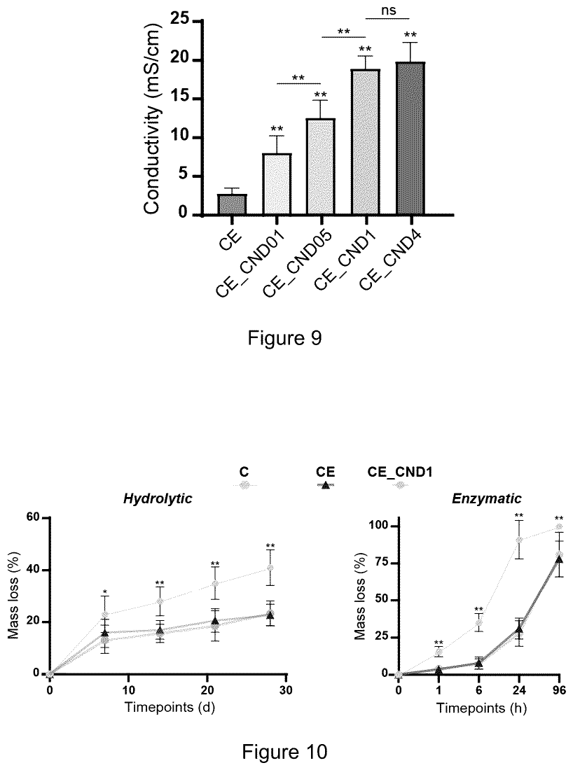

Successively, non-contact Atomic Force Microscopy (AFM) was employed to visualize the GlyCND spatial arrangement within the collagen matrix (FIG. 7), characterized by randomly distributed clusters of the carbon nanodots. The AFM linear depth profiles (FIG. 8) confirm a size distribution of the GlyCNDs ranging from ˜6 to 16 nm, as previously reported by Naccache and co-authors.⁴² Conductivity measurements assessed how the incorporation of GlyCNDs into the collagen matrix modulates the overall electrical properties of the nanocomposite. To this end, different GlyCND concentrations, ranging from 0.1 to 4.0 mg/ml, were tested and the resulting conductivity was compared to the one displayed by CE. As shown in FIG. 9, the relatively poor conductivity of CE (2.8 mS/cm) significantly increases as a result of the addition of the GlyCNDs up to 1 mg/ml. Interestingly, a higher concentration (i.e., 4 mg/ml) does not result in additional changes. The inventors hypothesize that concentrations higher than 1 mg/ml saturate the availability of free NH2 groups, thereby resulting in unreacted GlyCNDs that ultimately become suspended and thus do not contribute to the overall conductivity. Finally, we evaluated the chemical stability of the nanocomposite.

In accordance with results already reported in the literature,^{44,45} the inventors found that the presence of EDC/NHS greatly improves the resistance to both hydrolytic (FIG. 10, left) and enzymatic (FIG. 10, right) degradation of pristine collagen (hereafter referred to as C). Both CE and CE_CND1 conditions display a significantly lower mass loss due to hydrolytic degradation when compared to C. The same consideration is also valid for enzymatic degradation in the presence of collagenase IV. This increased stability of the CE_CND1 condition highlights the potential of our novel nanocomposite to overcome one of the major limitations of collagen hydrogels for tissue engineering and neural applications.

### Cytotoxicity Assays

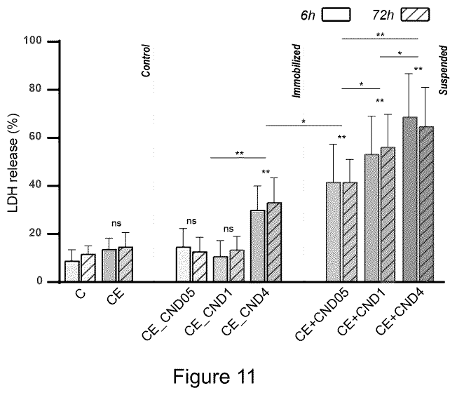

Despite the considerable efforts to characterize the potential neurotoxicity of carbon-based nanomaterials,⁴⁶⁻⁴⁸ the results reported in the literature remain controversial, likely due to interstudy variations in size, physicochemical properties and concentration. Here, to evaluate the cytocompatibility of both collagen-immobilized and suspended GlyCNDs with iPSCs-derived NP spheroids, we carried out a colorimetric LDH assay complemented with propidium iodide (PI) live staining of dead cells. Three different concentrations (i.e., 0.5 mg/ml, 1 mg/ml and 4 mg/ml) of GlyCNDs were both immobilized within the collagen matrix by EDC/NHS coupling (hereafter referred to as CE_CND05, CE_CND1, CE_CND4) and suspended in the culture medium (hereafter referred to as CE+CND05, CE+CND1, CE+CND4). Pristine collagen (C) and EDC/NHS crosslinked collagen (CE) were used as controls. FIG. 11 displays the percentage values of released LDH found for the different conditions at 6 and 72 h.

The results indicate that the viability of NC spheroids is not impacted by GlyCNDs immobilized within the collagen matrix at concentrations of 1 mg/ml or lower. In fact, the percentage values of released LDH of CE_CND05 and CE_CND1 are not statistically different from the ones displayed by the control groups at both time intervals. Conversely, we observed that CE_CND4 significantly increases cell mortality at both time points, with LDH percentage values that doubled in comparison to those displayed by the controls. When GlyCNDs are suspended in the culture medium, significantly higher levels of LDH are observed for both time points. In particular, the cytotoxicity increases proportionally with the GlyCNDs concentration, with CE+CND4 displaying the highest LDH release values.

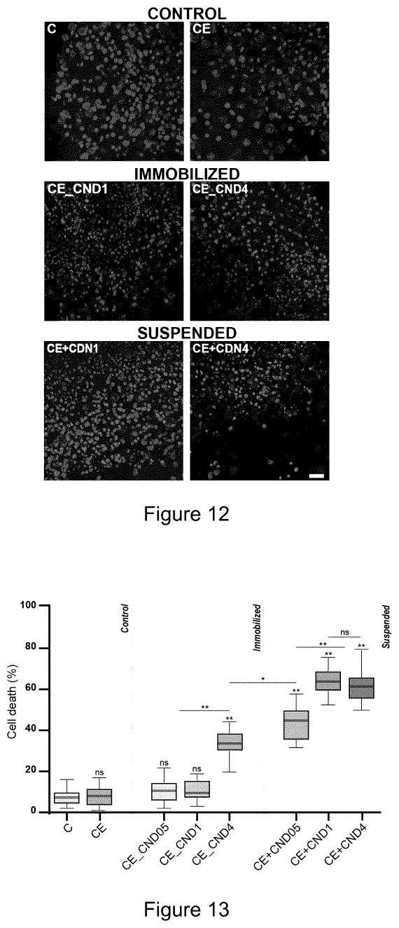

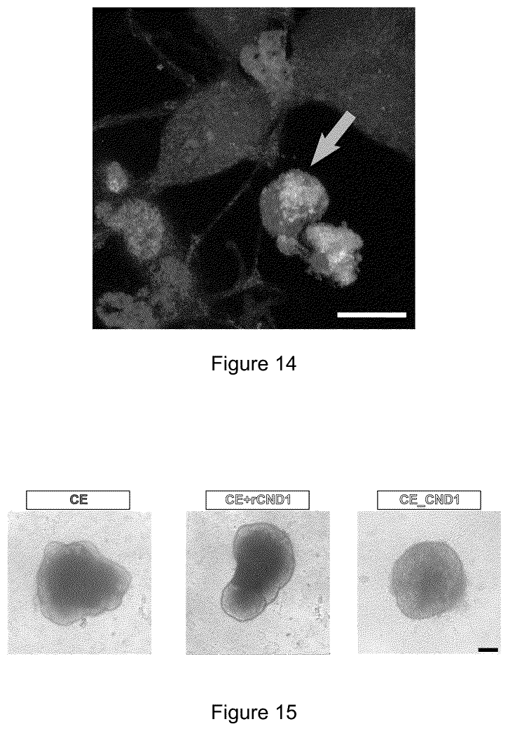

These results were further complemented by PI staining of the NP spheroids at 72 h. FIG. 12 displays representative images of controls, immobilized and suspended GlyCNDs conditions. It is evident that NP spheroids cultured in the GlyCNDs-supplemented medium display significantly higher amounts of dead cells compared to both controls and collagen with immobilized GlyCNDs. Cell death percentage (FIG. 13) was calculated as the PI-positive (PI+) cells over the total amount of nuclei. Not surprisingly, PI staining mirrors the results obtained with the LDH assay. Immobilized GlyCNDs display similar cell death percentages to control groups, while suspended GlyCNDs yield higher cytotoxicity. Overall, our results provide additional evidence to support recent arguments that emphasize the need for immobilization of carbon-based nanomaterials within scaffolds to mitigate undesirable outcomes that are associated with their use as suspended particles, such as bioaccumulation (FIG. 14).^{32,49}

Furthermore, the consistent LDH values found at 6 and 72 h across all conditions with suspended GlyCNDs indicate cell death onset within the first 6 h of exposure. Most importantly, the cytotoxicity assays played a crucial role in determining that immobilized GlyCNDs at a concentration of 1 mg/ml displayed viability results that were comparable to those of the control groups, while greatly improving the poor electrical conductivity of pristine collagen. For this reason, the inventors selected CE_CND1 as the experimental condition for the cell studies presented in the following sections.

### Neurite Growth and Formation of a Complex Neuronal Network

One of the primary goals of neural tissue engineering is to develop functional scaffolds that favor neurite spreading and extension while supporting the establishment of complex neural networks. After only 5 days in culture, several neurites spreading from spheroids embedded in the CE_CND1 nanocomposite are visible, while none/few are observed in control conditions (i.e., CE and CE+rCND1) (FIG. 15). To quantitatively characterize morphological differences, the spheroids were stained for β III-tubulin and imaged via confocal microscopy at 14 days.

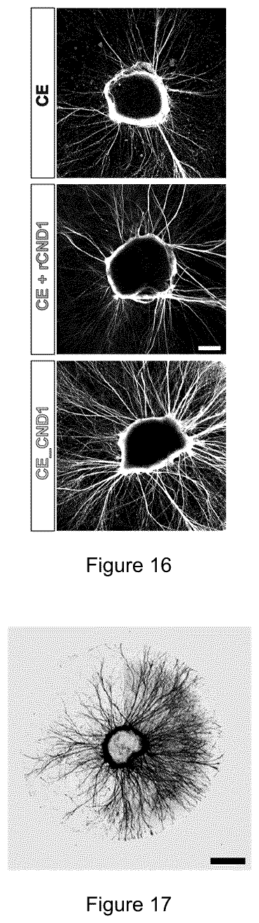

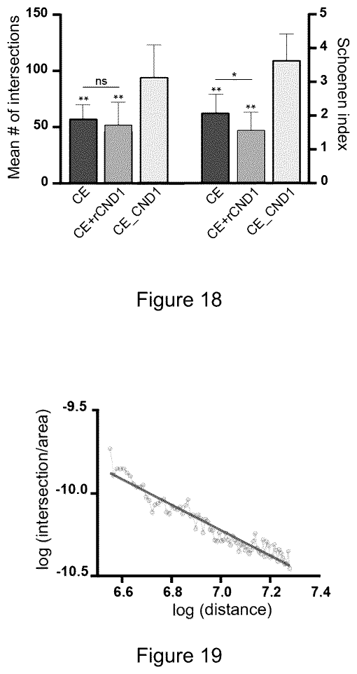

The CE_CND1 promotes a significantly more extensive neurite outgrowth when compared to CE and CE+rCND1 (FIG. 16). The overall branching complexity was evaluated using a semi-automated Sholl analysis centered on the spheroid's body. In particular, the total number of neurite intersections was calculated using 20 μm-spaced concentrical hemispheres (FIG. 17). As shown in FIG. 18, spheroids within the CE_CND1 condition exhibit an average number of neurite intersections of 95±28, which is significantly higher than when they are placed within CE (58±21) and CE+rCND1 (47±18) matrices.

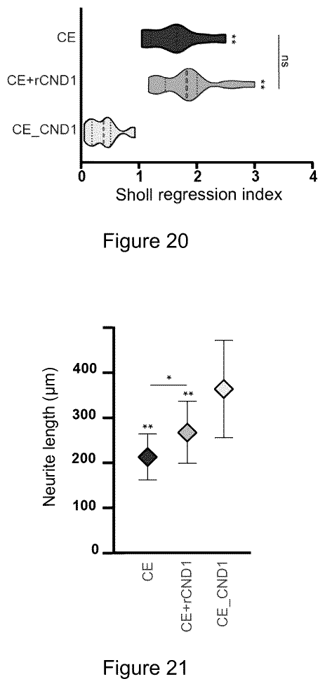

To evaluate the neurite ramification tendency, we utilized the Schoenen index, defined as the maximum number of intersections divided by the number of primary branches exiting from the spheroid's soma. As shown in FIG. 18, the CE_CND1 condition displays the highest average index of 3.6±0.8. Interestingly, the significantly lower average value found for spheroids in the CE+rCND1 condition when compared to CE (i.e., 1.7 vs 2.1), indicates that the released GlyCNDs have detrimental effects on the formation of a complex neuronal network, thereby confirming previous similar observations with carbon nanomaterials.^{20,22-25} The overall increased branching complexity of spheroids in CE_CND1 is also validated by the Sholl's regression index, a parameter that quantifies the rate of decay of the number of branches with distance from the center of analysis (FIG. 19). As shown in FIG. 20, the CE_CND1 condition is characterized by the lowest index (0.3) when compared to that of the CE (1.6) and CE+rCND1 (1.8) matrices.

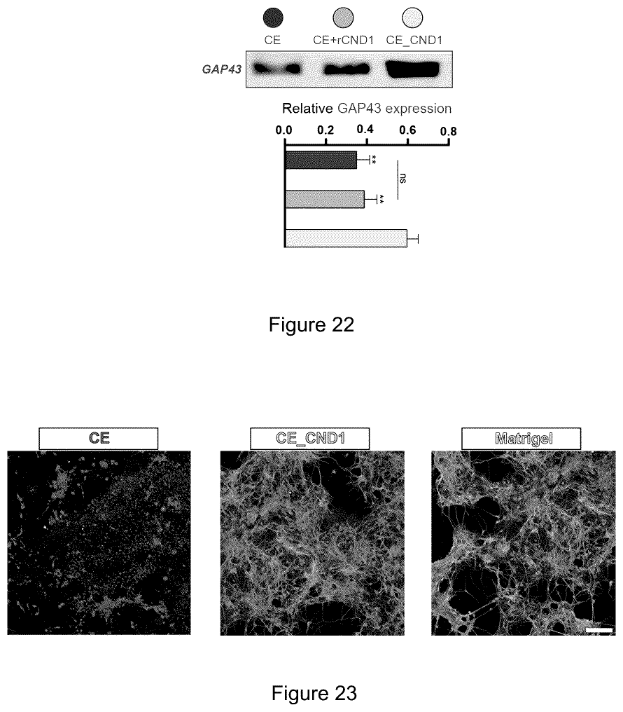

In addition to the increased neural network complexity, spheroids embedded in the CE_CND1 also display the highest neurite length (FIG. 21) of 369±113 μm. Interestingly, in the CE+rCND1 matrix, neurites are significantly longer when compared to the CE condition (i.e., 273±78 μm vs 207±53 μm). To unveil the underlying mechanisms that drive such divergent morphology, we investigated via Western blotting the expression of growth associated protein 43 (GAP43), a crucial protein for neuritogenesis.⁵⁰⁻⁵² As shown in FIG. 22, spheroids in CE_CND1 express a significantly higher amount of GAP43 at 14 days compared to those within the control conditions. Taken together, our findings reveal that immobilized GlyCNDs upregulated NP spheroid expression of GAP43, which is decisive in neurite outgrowth and complex neural network formation. In contrast, although GlyCNDs released in solution facilitate the development of longer neurites, they exhibited significantly lower ramifications than NP spheroids embedded in CE.

To investigate whether the ability of the nanocomposite to support neuritogenesis extends to mature neurons, the inventors used the CE_CND1 nanocomposite as a matrix for a scaffold-based 3D culture of mouse primary cortical neurons, confirming that the establishment of a complex neural network is observed within 7 days (FIG. 23). Compared to CE, cortical neurons grown in the CE_CND1 matrix show a significant increase in neurite outgrowth that enabled the formation of intricate networks. Notably, the morphology elicited by CE_CND1 was comparable to the one observed in Matrigel, the gold standard matrix in neurobiology. 53 The use of a different cell type offers a compelling validation of the nanocomposite's distinctive ability to support key processes associated with neuroregeneration.

The consistency of results between NP spheroids and primary cortical neurons supports in fact the evidence that the nanocomposite's beneficial effects transcend specific cell types and in vitro testing platforms. In particular, both spheroids and the 3D culture of primary neurons emulate more closely than conventional 2D monolayer systems the in vivo cellular environment, thereby providing a physiologically relevant representation of neuron biomaterial interactions. Taken together, our findings highlight the potential wide-ranging applicability of the CE_CND1 nanocomposite for reconstruction of lost neuroanatomical connectivity.

### Neuronal Differentiation

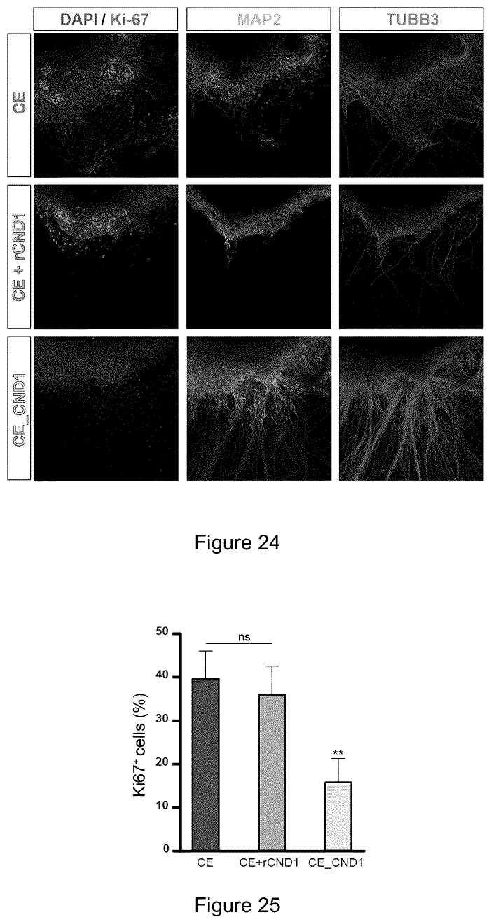

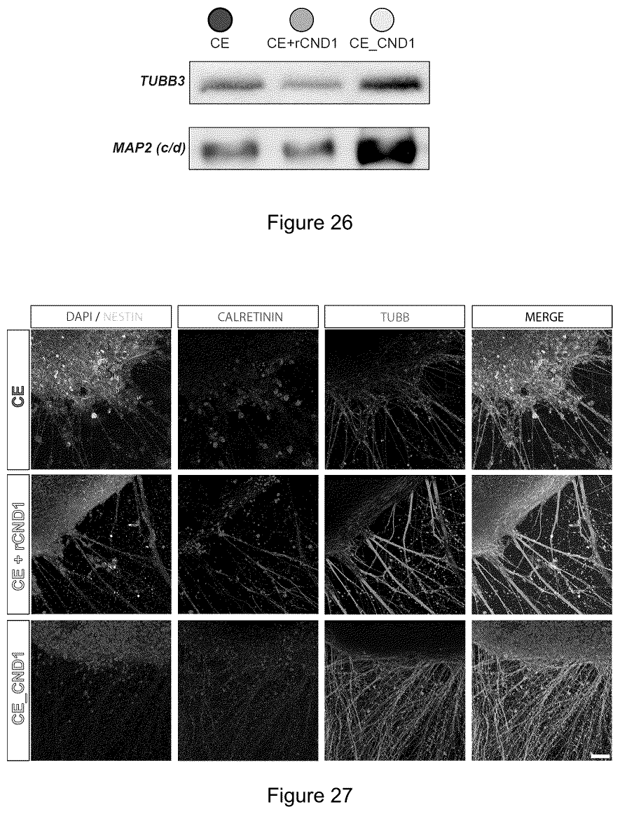

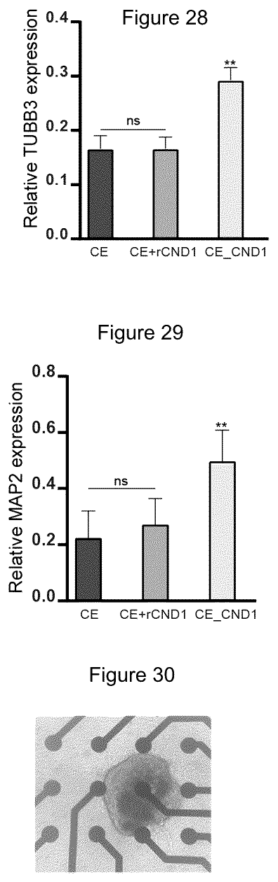

To expand the breadth of the investigation of the beneficial effects of the nanocomposite, we evaluated the expression levels of key neuronal maturation markers by immunofluorescence (IF) imaging and Western blotting. FIG. 24 displays representative IF images of NP spheroids embedded in CE, CE+rCND1 and CE_CND1 after 2 weeks of culture. Samples were stained for Ki-67, a nuclear proliferation marker,⁴ as well as for β III-tubulin and MAP2, an early and a mature neuronal marker, respectively. 55-57 The overall expression of these proteins indicates that the differentiation of the neurospheres embedded in the CE_CND1 matrix is promoted, showing significantly lower amounts of Ki-67-positive cells (FIG. 25) and higher levels of β III-tubulin and MAP2 (FIG. 26). These findings are also corroborated by the relative expression of calretinin (an early neuronal marker) and nestin (a neural progenitor marker), 57-59 In particular, spheroids cultured in CE and CE+rCND1 matrices display a higher amount of nestin when compared to the ones embedded in CE_CND1, which instead express higher levels of calretinin (FIG. 27). Quantitative Western blot analysis confirms IF imaging results by showing a higher relative expression of β III-tubulin (FIG. 28) and MAP2 (FIG. 29) for neurospheres in CE_CND1 when compared to CE and CE+rCND1.

Taken together, our findings indicate that the immobilized GlyCNDs induce a rapid neuronal differentiation and maturation of NP spheroids. This evidence is supported by the fact that the nanoparticles released from the matrix (CE_rCND1) is do not elicit any effect on differentiation, similar to the condition where they are absent (CE). The positive effects of immobilized GlyCNDs strengthen the potential and impact of carbon-based nanomaterials in neural tissue engineering. Specifically, the accelerated neuritogenesis and the elevated expression of the mature neuronal marker MAP2 highlight the remarkable capacity of our nanocomposite to accelerate neuronal differentiation of NPs (compared to previous literature on miPSCs)⁶⁰⁻⁶² and ensure their complete electrophysiological maturation, two crucial aspects for biomaterial-driven neural development and regeneration.

### Electrophysiological Maturation and Neuronal Network Communication

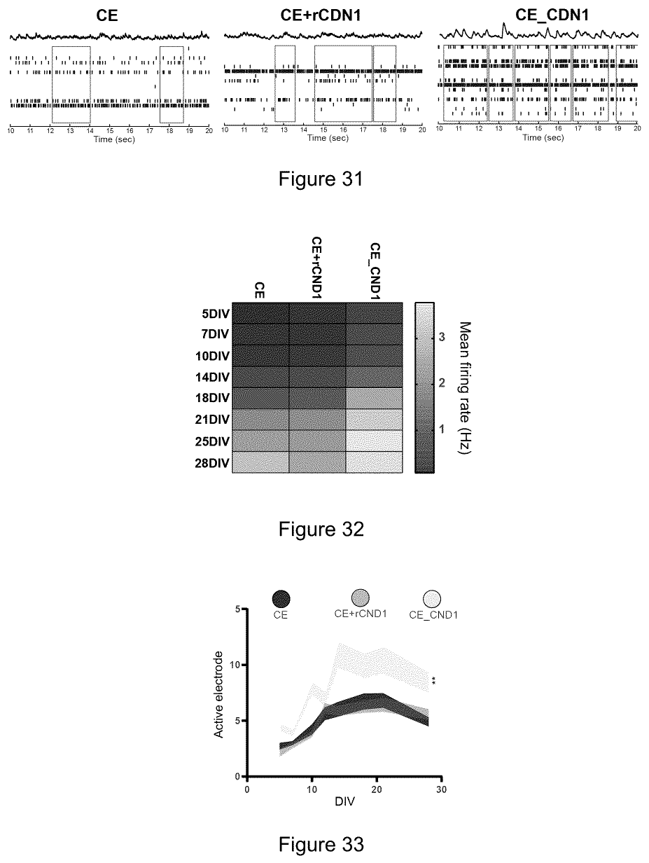

The inventors evaluated the spontaneous electrical activity of NP spheroids, both at the single electrode and network level, by capitalizing on commercially available multi-electrode arrays (MEAs) (FIG. 30). Starting at day 5 and consistently throughout the entire experimental duration (28 DIV), NP spheroids in the CE_CND1 matrix exhibit a remarkable enhancement in single-electrode activity. Specifically, the firing rate, which reflects the frequency of action potentials, is significantly higher than the other experimental groups (FIGS. 31, 32).

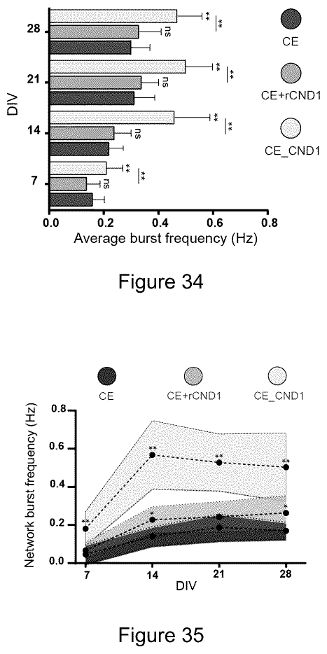

Furthermore, an increased number of active electrodes (FIG. 33) and an enhanced burst frequency can be observed (FIG. 34), indicating an increased synchronized activity among the cells in the spheroids. In addition to the single-electrode level, the CE_CND1 nanocomposite also positively influenced the network burst frequency, a measure of large-scale synchronized activity among different electrodes, which is consistently and significantly higher for the spheroids embedded in CE_CND1 (FIG. 35). This result indicates that the CE_CND1 nanocomposite facilitates the formation of functional connections among cells, yielding a more integrated neuronal network.

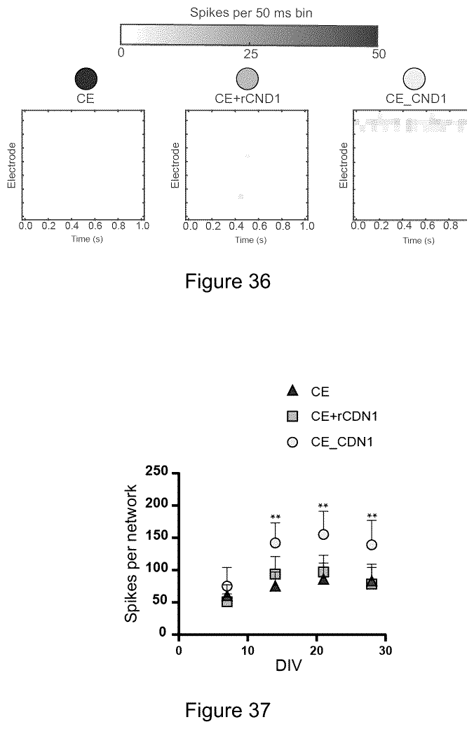

In addition, the average number of spikes per network burst is notably higher (FIGS. 36, 37), suggesting that the spheroids cultured in the nanocomposite established more complex and intensified network communications. By day 14, neurospheres show a remarkable single-electrode activity and the generation of intricate neuronal network connections. The importance of immobilizing the GlyCNDs within the collagen matrix is further demonstrated by the electrophysiological recordings relative to the CE_rCND experimental group. In fact, released GlyCNDs did not elicit noticeable or consistent alterations from the electrical signature that was displayed by CE, neither at the single electrode or at the network level.

These findings demonstrate that collagen-immobilized GlyCNDs provide a favorable microenvironment that accelerates the electrophysiological maturation and the establishment of effective neural network formation. Together with the neuronal differentiation and the morphogenesis data reported in the previous sections, these results demonstrate that the electroconductive nanocomposite enhances the maturation of functionally active neuronal cells and promotes their communication through an intensified neural network.

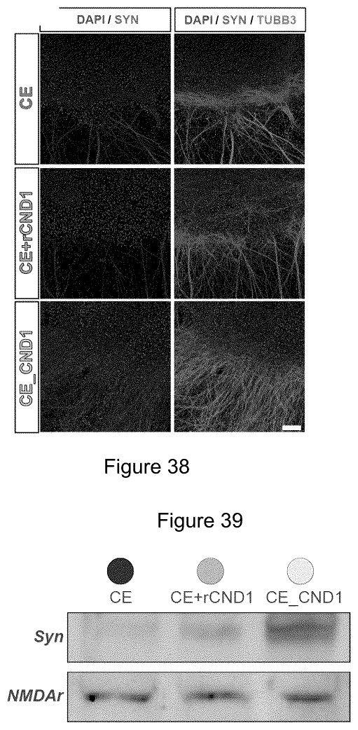

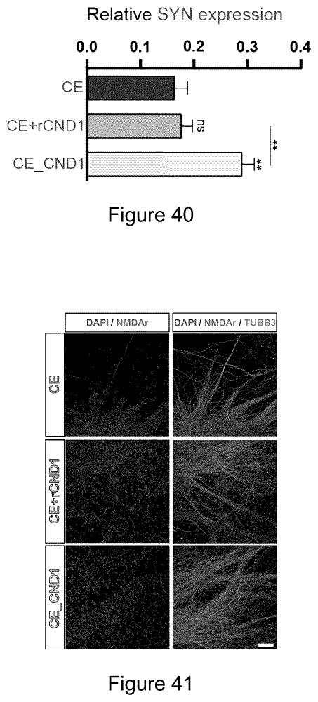

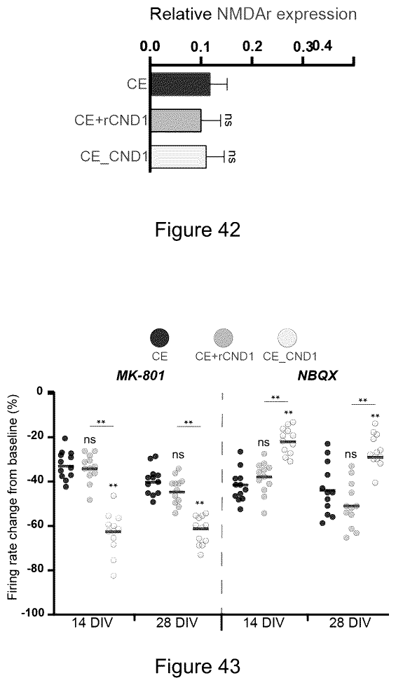

To gain a deeper understanding of the underlying mechanisms that contribute to such enhanced electrophysiological activity, we investigated by immunofluorescence and Western blotting the expression of synapsin (i.e., a crucial phosphoprotein known to promote the establishment and maintenance of synaptic connections by actively regulating the release of neurotransmitters) and that of NMDA receptors (NMDAr), which are primarily involved in the transmission of excitatory information.⁶³⁻⁶⁶ FIGS. 38-40 show that NP spheroids embedded in CE_CND1 exhibit higher levels of synapsin, namely 0.27 versus 0.16 and 0.18 for the CE and CE_rCND1 groups, respectively. On the other hand, NMDA receptor levels are comparable for the conditions tested (FIGS. 41, 42). However, acute treatment with MK-801, a noncompetitive NMDA receptor antagonist, significantly affects the electrical activity; in particular that of the spheroids embedded in the CE_CND1 nanocomposite.

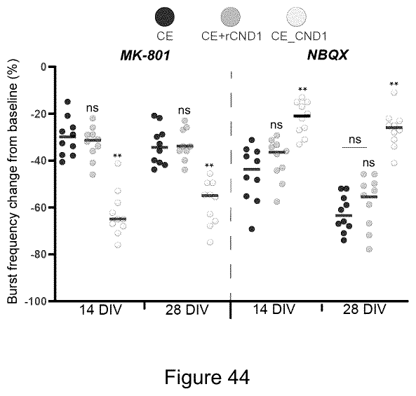

As depicted in FIG. 43, upon treatment with MK801 on day 14 and 28, the firing rate exhibits a reduction of 63% and 61%, respectively, compared to the spontaneous baseline recording. Furthermore, MK-801 acute treatments are also found to reduce the network activity (FIG. 44). Notably, the impact of NMDAr antagonist is significantly more severe on the firing rate and network burst of the spheroids embedded in CE_CND1, indicating an overall activity that is heavily dependent on the availability of NMDA receptors. In comparison, acute treatment with NBQX, a commonly used AMPA and kainate receptor blocker, yields the opposite trend, whereby neurospheres in CE_CND1 are significantly less susceptible to AMPA receptor antagonist than those in the CE and CD_rCND1 matrices at both 14 and 28 days.

This differential impact of the two antagonists suggests that the electrophysiological activity of NP spheroids is mainly governed by NMDAr activation. Based on these findings, we hypothesize the presence of direct interactions between the GlyCNDs in the CE_CND1 and NMDAr alters the channel dynamics and ultimately leads to channel activation. Although this theory needs further scrutiny since other surface receptors (e.g., neurotrophin and nerve growth factor) can regulate NMDAr dependent currents,⁶⁷ our findings nonetheless indicate biomaterial-derived effects on the NMDA receptors which may, either completely or in part, explain the reported cellular effects, as NMDAr activation is known to drive neurite growth, neural differentiation and maturation as well as electrical activity.

### CONCLUSION

In conclusion, this disclosure provides a novel electroconductive nanocomposite consisting of a collagen type I matrix decorated with GlyCNDs, ultimately demonstrating its promising potential as a biomaterial for applications ranging from neural tissue engineering and neuroregenerative medicine to bioinks and matrices for 3D cultures and biomimetic in vitro models. In particular, after establishing an effective anchorage method for GlyCNDs within the collagen matrix through EDC-NHS coupling, we carried out a cytotoxicity assay to inform the optimization of GlyCNDs concentration towards a biocompatibility comparable to that of pristine collagen. The biological characterization of the nanocomposite was carried out with mouse iPSCs-derived NP spheroids (and, in part, with a 3D culture of primary neurons) for a more physiologically accurate representation of in vivo conditions towards enhancing the reliability and translatability of our findings.

The CE_CND1 nanocomposite substantially enhances the neuronal differentiation of NP spheroids and promotes neuritogenesis, with conspicuous dendritic arborization and axonal outgrowth, ultimately facilitating the formation of functional and highly integrated neural networks. Furthermore, acute MK-801 treatment suggests a direct interaction between collagen-immobilized GlyCNDs and post-synaptic NMDA receptors. Results from this work thus provide the fundamental knowledge for a new biomaterial-based tissue engineering strategy for the treatment of neuronal disorders via the restoration of lost neurons and neuroanatomical connectivity, thereby aiming to address the key pathological substrate of these pathologies and ultimately improving the life of millions of patients worldwide.

### EMBODIMENTS

In an embodiment there is provided an injectable, aligneable and electroconductive hydrogel-nanodot nanocomposite material, comprising:
a physiologically acceptable polymer comprised of polymer chains/fibers with characteristic functional groups R″ along the polymer chains/fibers;
electrically conductive nanodots bearing preselected functional groups R′ decorating their outer surface; and
crosslinker agents having opposed ends and being bound at one end thereof to the functional groups R″ on the polymer chains/fibers and bound at the other end thereof to functional groups R′ on the outer surface of the electrically conductive nanodots, via covalent, electrostatic, or cathecol-based interactions.

In an embodiment there is provided a method of producing an injectable, aligneable and electroconductive hydrogel-nanodot nanocomposite material, comprising:
providing a physiologically acceptable polymer comprised of polymer chains/fibers forming a polymer matrix of the composite, functionalizing the polymer chains/fibers with preselected functional groups R″ attached along the polymer chains/fibers to produce functionalized polymer chains/fibers;
providing electrically conductive nanodots and functionalizing the electrically conductive nanodots with preselected functional groups R′ to an outer surface of the electrically conductive nanodots to producing functionalized electrically conductive nanodots; and
mixing the functionalized electrically conductive nanodots and the functionalized polymer strands with crosslinker molecules having opposed ends under conditions suitable induce formation of cross linkages between the electrically conductive nanodots and polymeric matrix through the crosslinker molecules having one end thereof bound to the R″ functional groups on the polymer strands and the other end of the crosslinker molecules bound to the R′ functional groups on the electrically conductive nanodots.

In an embodiment the functional groups R″ are any one or combination of NH₂, NH, NCO, CHs, CH₂, COOH, CO, OH, SH, SO₃, H and O, and
wherein the functional groups R′ are any one or combination of NH₂, COOH, NH, OH, SH, O, SiOH, PO₃H₂, SO₃ and CONH₂.

In an embodiment the physiologically acceptable polymer is any one or combination of synthetic polymers and natural polymers.

In an embodiment the natural polymers include any one or combination of collagen, agarose, gelatine, fibrin, elastin cellulose, silk fibroin, chitin, chitosan, glycosaminoglycans, keratin, pectin, hyaluronic acid, lactate, starch and lignin.

In an embodiment the synthetic polymers include any one or combination of collagen, Polyethylene (PE), Polypropylene (PP), Polycarbonate (PC), Polyimide (PI), Polystyrene (PS), Poly(lactic-co-glycolic acid) (PLGA), Polylactic acid (PLA), Poly(lactic acid)-graft-poly(methacrylic acid) (PLA-g-P(MAA)), Poly-L-lysine (PLL), Polyethylene terephthalate (PET), Gelatin methacryloyl (GelMA), Polyethylene glycol diacrylate (PEGDA), Polyethylene glycol (PEG), Polyethylene oxide (PEO), Polyvinyl chloride (PVC), Polymethyl methacrylate (PMMA), Polytetrafluoroethylene (PTFE), Polycaprolactone (PCL), Polyvinyl alcohol (PVA), Polydimethylsiloxane (PDMS), Polyether ether ketone (PEEK), Polyhydroxyalkanoate (PHA)

In an embodiment the electrically conductive nanodots are any one or combination of carbon nanodots, graphene nanodots, metallic nanodots, semiconductor nanodots, silicon nanodots, indium tin oxide nanodots, copper nanodots and zinc oxide nanodots.

In an embodiment the electrically conductive nanodots have a diameter in a range from about 1 nm to about 100 nanometers.

In an embodiment the crosslinker molecules are bound to the functional groups R′ and R″ by any one or combination of covalent bonding, electrostatic and cathecol-based interactions.

In an embodiment the crosslinker agents are any one or combination of molecules selected on the basis of their ability for creating permanent covalent chemistries, dynamic covalent chemistries, ions capable of creating electrostatic interactions, and molecules for other classes of interactions.

In an embodiment the crosslinker agents are any one or combination of
the molecules selected on the basis of their ability for creating permanent covalent chemistries including amine, amide, urea, thioether, siloxane and acrylamide bonds,
the molecules selected on the basis of their ability for creating dynamic covalent chemistries including imine, acylhydrazone, oxime,boronic-ester, disulfide and thioester bonds,
ions selected on the basis of their ability for creating electrostatic interactions including cation-anion, bridging ion, cation-π, anion-π, π-π interactions and hydrogen bonding,
and the molecules for other classes of interactions including cyclodextrin-based molecules, cathecol-based molecules and cucurbit[n]uril-based molecules.

The foregoing description of the preferred embodiments of the invention has been presented to illustrate the principles of the invention and not to limit the invention to the particular embodiment illustrated. It is intended that the scope of the invention be defined by all of the embodiments encompassed within the following claims and their equivalents.

### REFERENCES

1. Elena Martinez JPSP& FV. Advanced bioengineering technologies for preclinical research. Adv Phys. 2019; 4(1). doi:10.1080/23746149.2019.1622451
2. Centeno, E. G. Z., Cimarosti, H. & Bithell A. 2D versus 3D human induced pluripotent stem cell-derived cultures for neurodegenerative disease modelling. Mol Neurodegener. 2018; 13.
3. Rathore, R. S., R Ayyannan, S. & Mahto S K. Emerging three-dimensional neuronal culture assays for neurotherapeutics drug discovery. Expert Opin Drug Discov. 2022; 17.
4. Ozgun A., Lomboni D., Arnott H., Staines, W. A., Woulfe J. VF. Biomaterials-based strategies for in vitro neural models. Biomater Sci. 2022; 10(5):1134-1165.
5. Guo, X., Tang, L. & Tang X. Current Developments in Cell Replacement Therapy for Parkinson's Disease. Neuroscience. Published online 2021.
6. Wenker, S. D. & Pitossi F J. Cell therapy for Parkinson's disease is coming of age: current challenges and future prospects with a focus on immunomodulation. Gene Ther. 2020; 27.
7. Elsworth J D. Parkinson's disease treatment: past, present and future. J Neural Transm. 2020; 127.
8. GordiAn-Vélez W J et al. Restoring lost nigrostriatal fibers in Parkinson's disease based on clinically-inspired design criteria. Brain Res Bull. 2021; 175.
9. Doblado, L. R., Martinez-Ramos, C. & Pradas M M. Biomaterials for Neural Tissue Engineering. Front Nanotechnol. 2021; 3.
10. Gu X. Progress and perspectives of neural tissue engineering. Front Med. 2015; 9.
11. Boni R, Ali A, Shavandi A, Clarkson A N. Current and novel polymeric biomaterials for neural tissue engineering. J Biomed Sci. 2018; 25(1):1-21. doi:10.1186/s12929-018-0491-8
12. Roth J G et al. Advancing models of neural development with biomaterials. Nat Rev Neurosci. 2022; 22.
13. Liu, Z., Wan, X., Wang, Z. L. & Li L. Electroactive Biomaterials and Systems for Cell Fate Determination and Tissue Regeneration: Design and Applications. Adv Mater. 20221; 33.
14. Melrose, J., Hayes, A. J. & Bix G. The CNS/PNS extracellular matrix provides instructive guidance cues to neural cells and neuroregulatory proteins in neural development and repair. Int J Mol Sci. 2021; 22.
15. Gao, C., Song, S., Lv, Y., Huang, J. & Zhang Z. Recent Development of Conductive Hydrogels for Tissue Engineering: Review and Perspective. Macromol Biosci. 2022; 22.
16. Liu H et al. An Electroconductive Hydrogel Scaffold with Injectability and Biodegradability to Manipulate Neural Stem Cells for Enhancing Spinal Cord Injury Repair. Biomacromolecules. 2023; 24.
17. Yang S et al. Polypyrrole/Alginate Hybrid Hydrogels: Electrically Conductive and Soft Biomaterials for Human Mesenchymal Stem Cell Culture and Potential Neural Tissue Engineering Applications. Macromol Biosci 1. 2016; 16.
18. Xu X et al. Conductive Collagen-Based Hydrogel Combined With Electrical Stimulation to Promote Neural Stem Cell Proliferation and Differentiation. Front Bioeng Biotechnol. 2022; 10.
19. Samadian, H., Khastar, H. E. Bioengineered 3D nanocomposite based on gold nanoparticles and gelatin nanofibers for bone regeneration: in vitro and in vivo study. Sci Rep. 2021; 11.
20. Carlos Redondo-Gomez, Rocio Leandro-Mora, D. B.-B. & Christopher Espinoza-Araya D, Hidalgo-Barrantes and JVB. Recent Advances in Carbon Nanotubes for Nervous Tissue Regeneration. Adv Polym Technol. Published online 2020.
21. Barrejón M et al. TEGylated Double-Walled Carbon Nanotubes as Platforms to Engineer Neuronal Networks. ACS Appl Mater Interfaces. Published online 2022.
22. Ye L et al. Carbon Nanotube-Hydrogel Composites Facilitate Neuronal Differentiation While Maintaining Homeostasis of Network Activity. Adv Mater. 2021; 33.
23. Anirban A. Fuzzy graphene for neuron control. Nat Rev Phys. 2020; 2.
24. Lawkowska K et al. Application of graphene in tissue engineering of the nervous system. J Mol Sci. 2022; 23.
25. Kenry, Lee, W. C., Loh, K. P. & Lim C T. When stem cells meet graphene: Opportunities and challenges in regenerative medicine. Biomaterials. 2018; 155(236-250).
26. Ucar, B. & Humpel C. Collagen for brain repair: Therapeutic perspectives. Neural Regen Res. 2018; 13:595-598.
27. Kumar, R., Kumar, V. B. & Gedanken A. Sonochemical synthesis of carbon dots, mechanism, effect of parameters, and catalytic, energy, biomedical and tissue engineering applications. Ultrason Sonochem. 2020; (105009).
28. Cali, F., Fichera, L. & Tuccitto N. Comprehensive step-by-step procedure to setup a molecular communication through liquid experiment. MethodsX. 2022; 9.
29. Pas S P. The rise of three-dimensional human brain cultures. Nature. 2018; 553.
30. Castellanos-Montiel M J et al. An Optimized Workflow to Generate and Characterize iPSCDerived Motor Neuron (MN) Spheroids. Cells. 2023; 12.
31. Brüningk, S. C., Rivens, I., Box, C., Oelfke, U. & ter Haar G. 3D tumour spheroids for the prediction of the effects of radiation and hyperthermia treatments. Sci Rep. 2020; 10.
32. Lee H et al. Effect of carbon nanomaterial dimension on the functional activity and degeneration of neurons. Biomaterials. 2021; 279.
33. Kang K S et al. In-vitro cytotoxicity assessment of carbon-nanodot-conjugated Fe-aminoclay (CD-FeAC) and its bio-imaging applications. J Nanobiotechnology. 2015; 13.
34. Chan M H et al. Natural carbon nanodots: Toxicity assessment and theranostic biological application. Pharmaceutics. 2021; 13.
35. Martynas Talaikis, Simona Strazdaité, Mantas Žiaunys GN. Far-Off Resonance: Multiwavelength Raman Spectroscopy Probing Amide Bands of Amyloid-β-(37-42) Peptide. Molecules. 2020; 25(15).
36. Gullekson, C., Lucas, L., Hewitt, K. & Kreplak L. Surface-sensitive Raman spectroscopy of collagen I fibrils. Biophys J. 2011; 100.
37. Bonifacio, A. & Sergo V. Effects of sample orientation in Raman microspectroscopy of collagen fibers and their impact on the interpretation of the amide 111 band. Vib Spectrosc. 2010; 53.
38. Acri G et al. Preliminary study for the application of Raman spectroscopy for the identification of Leishmania infected dogs. Sci Rep. Published online 2022.
39. Nguyen T T et al. Characterization of type i and IV collagens by Raman microspectroscopy: Identification of spectral markers of the dermo-epidermal junction. Spectrosc (New York). 2012; 27.
40. Schindelin J et al. Fiji: An open-source platform for biological-image analysis. Nat Methods. 2012; 9.
41. Arshadi, C., Günther, U., Eddison, M., Harrington, K. I. S. & Ferreira T A. SNT: a unifying toolbox for quantification of neuronal anatomy. Nat Methods. 2021; 18.
42. Macina, A., De Medeiros, T. V. & Naccache R. A carbon dot-catalyzed transesterification reaction for the production of biodiesel. J Mater Chem A. 2019; 7.
43. Macina, A., De Medeiros, T. V. & Naccache R. A carbon dot-catalyzed transesterification reaction for the production of biodiesel. J Mater Chem A. Published online 2019.
44. Nong L M et al. The effect of different cross-linking conditions of EDC/NHS on type II collagen scaffolds: an in vitro evaluation. Cell Tissue Bank. 2019; 20.
45. Nair, M., Johal, R. K., Hamaia, S. W., Best, S. M. & Cameron R E. Tunable bioactivity and mechanics of collagen-based tissue engineering constructs: A comparison of EDC-NHS, genipin and TG2 crosslinkers. Biomaterials. Published online 2020.
46. Kharlamova, M. V & Kramberger C. Cytotoxicity of Carbon Nanotubes, Graphene, Fullerenes, and Dots. Published online 2023.
47. Zakrzewska K E et al. Analysis of the cytotoxicity of carbon-based nanoparticles, diamond and graphite, in human glioblastoma and hepatoma cell lines. PLoS One. 2015; 10.
48. Yuan, X., Zhang, X., Sun, L., Wei, Y. & Wei X. Cellular Toxicity and Immunological Effects of Carbon-based Nanomaterials. Part Fibre Toxicol. 2019; 16.
49. Devasena T et al. Insights on the Dynamics and Toxicity of Nanoparticles in Environmental Matrices. Bioinorg Chem Appl. Published online 2022.
50. Zhang, C., Cui, L., He, W., Zhang, X. & Liu H. DI-3-n-butylphthalide promotes neurite outgrowth of primary cortical neurons by Sonic Hedgehog signaling via upregulating Gap43. Exp Cell Res. 2021; 398.
51. Wang Z et al. PEITC promotes neurite growth in primary sensory neurons via the miR-17-5p/STAT3/GAP-43 axis. J Drug Target. 2019; 27.
52. Holahan M R. A shift from a pivotal to supporting role for the growth-associated protein (GAP-43) in the coordination of axonal structural and functional plasticity. Front Cell Neurosci. 2017; 11.
53. Fang, Y. & Eglen R M. Three-Dimensional Cell Cultures in Drug Discovery and Development. SLAS Discov. 2017; 22.
54. Dalgin G et al. Developmental defects and impaired network excitability in a cerebral organoid model of KCNJ11 p.V59M-related neonatal diabetes. Sci Rep. 2021; 11.
55. Santos A K et al. Mouse Neural Stem Cell Differentiation and Human Adipose Mesenchymal Stem Cell Transdifferentiation Into Neuron- and Oligodendrocyte-like Cells With Myelination Potential. Stem Cell Rev Reports. 2022; 18.
56. Park S Y et al. Human Mesenchymal Stem Cell-Derived Extracellular Vesicles Promote Neural Differentiation of Neural Progenitor Cells. Int J Mol Sci. Published online 2022.
57. Hao M et al. Multifunctional Hydroxyapatite Nanobelt Haystacks Integrated Neural Stem Cell Spheroid for Rapid Spinal Cord Injury Repair. Adv Funct Mater. Published online 2023.
58. Yao H et al. CHD7 promotes neural progenitor differentiation in embryonic stem cells via altered chromatin accessibility and nascent gene expression. Sci Rep. 2020; 10.
59. Szymańska-Chabowska, A., Świçtkowski, F., Jankowska-Polańiska, B., Mazur G& C, M. Nestin Expression as a Diagnostic and Prognostic Marker in Colorectal Cancer and Other Tumors. Clin Med Insights Oncol. 2021; 15.
60. Chung, C. Y., Yang, J. T. & Kuo Y C. Polybutylcyanoacrylate nanoparticle-mediated neurotrophin-3 gene delivery for differentiating iPS cells into neurons. Biomaterials. 2013; 34.
61. Armijo E et al. Induced pluripotent stem cell-derived neural precursors improve memory, synaptic and pathological abnormalities in a mouse model of alzheimer's disease. Cells. 2021; 10.
62. Klincumhom N et al. Generation of neuronal progenitor cells and neurons from mouse sleeping beauty transposon-generated induced pluripotent stem cells. Cell Reprogram. 2012; 14.
63. Rajani, V., Sengar, A. S. & Salter M W. Tripartite signalling by NMDA receptors. Front Neurosci. 2019; 13.
64. Liao M et al. 3D Ti3C2TxMXene-Matrigel with Electroacoustic Stimulation to Promote the Growth of Spiral Ganglion Neurons. ACS Nano. 2022; 16.
65. Liu, J., Chang, L., Song, Y., Li, H. & Wu Y. The role of NMDA receptors in Alzheimer's disease. Front Neurosci. 2019; 13.
66. Hartmann J et al. Alginate-Laminin Hydrogel Supports Long-Term Neuronal Activity in 3D Human Induced Pluripotent Stem Cell-Derived Neuronal Networks. Adv Mater Interfaces. 2023; 10.
67. Jarvis C R et al. Neurotrophin modulation of NMDA receptors in cultured murine and isolated rat neurons. J Neurophysiol. 1997; 78.

1. An injectable, aligneable and electroconductive hydrogel-nanodot nanocomposite material, comprising:
a physiologically acceptable polymer comprised of polymer chains/fibers with characteristic functional groups R″ along the polymer chains/fibers;
electrically conductive nanodots bearing preselected functional groups R′ decorating their outer surface; and
crosslinker agents having opposed ends and being bound at one end thereof to the functional groups R″ on the polymer chains/fibers and bound at the other end thereof to functional groups R′ on the outer surface of the electrically conductive nanodots, via covalent, electrostatic, or cathecol-based interactions.

2. The hydrogel-electroconductive nanodot nanocomposite material according to claim 1,
wherein the functional groups R″ are any one or combination of NH₂, NH, NCO, CH₃, CH₂, COOH, CO, OH, SH, SO₃, H and O, and
wherein the functional groups R′ are any one or combination of NH₂, COOH, NH, OH, SH, O, SiOH, PO₃H₂, SO₃ and CONH₂.

3. The hydrogel-electroconductive nanodot nanocomposite material according to claim 1, wherein the physiologically acceptable polymer is any one or combination of synthetic polymers and natural polymers.

4. The hydrogel-electroconductive nanodot nanocomposite material according to claim 3, wherein the natural polymers include any one or combination of collagen, agarose, gelatine, fibrin, elastin cellulose, silk fibroin, chitin, chitosan, glycosaminoglycans, keratin, pectin, hyaluronic acid, lactate, starch and lignin.

5. The hydrogel-electroconductive nanodot nanocomposite material according to claim 3, wherein the synthetic polymers include any one or combination of collagen, Polyethylene (PE), Polypropylene (PP), Polycarbonate (PC), Polyimide (PI), Polystyrene (PS), Poly(lactic-co-glycolic acid) (PLGA), Polylactic acid (PLA), Poly(lactic acid)-graft-poly(methacrylic acid) (PLA-g-P(MAA)), Poly-L-lysine (PLL), Polyethylene terephthalate (PET), Gelatin methacryloyl (GelMA), Polyethylene glycol diacrylate (PEGDA), Polyethylene glycol (PEG), Polyethylene oxide (PEO), Polyvinyl chloride (PVC), Polymethyl methacrylate (PMMA), Polytetrafluoroethylene (PTFE), Polycaprolactone (PCL), Polyvinyl alcohol (PVA), Polydimethylsiloxane (PDMS), Polyether ether ketone (PEEK), Polyhydroxyalkanoate (PHA).

6. The hydrogel-electroconductive nanodot nanocomposite material according to claim 1, wherein the electrically conductive nanodots are any one or combination of carbon nanodots, graphene nanodots, metallic nanodots, semiconductor nanodots, silicon nanodots, indium tin oxide nanodots, copper nanodots and zinc oxide nanodots.

7. The hydrogel-electroconductive nanodot nanocomposite material according to claim 1, wherein the electrically conductive nanodots have a diameter in a range from about 1 nm to about 100 nanometers.

8. The hydrogel-electroconductive nanodot nanocomposite material according to claim 1, wherein the crosslinker molecules are bound to the functional groups R′ and R″ by any one or combination of covalent bonding, electrostatic and cathecol-based interactions.

9. The hydrogel-electroconductive nanodot nanocomposite material according to claim 1, wherein the crosslinker agents are any one or combination of molecules selected on the basis of their ability for creating permanent covalent chemistries, dynamic covalent chemistries, ions capable of creating electrostatic interactions, and molecules for other classes of interactions.

10. The hydrogel-electroconductive nanodot nanocomposite material according to claim 9, wherein the crosslinker agents are any one or combination of
the molecules selected on the basis of their ability for creating permanent covalent chemistries including amine, amide, urea, thioether, siloxane and acrylamide bonds,
the molecules selected on the basis of their ability for creating dynamic covalent chemistries including imine, acylhydrazone, oxime,boronic-ester, disulfide and thioester bonds,
ions selected on the basis of their ability for creating electrostatic interactions including cation-anion, bridging ion, cation-π, anion-π, π-π interactions and hydrogen bonding,
and the molecules for other classes of interactions including cyclodextrin-based molecules, cathecol-based molecules and cucurbit[n]uril-based molecules.

11. A method of producing an injectable, aligneable and electroconductive hydrogel-nanodot nanocomposite material, comprising:
providing a physiologically acceptable polymer comprised of polymer chains/fibers forming a polymer matrix of the composite, functionalizing the polymer chains/fibers with preselected functional groups R″ attached along the polymer chains/fibers to produce functionalized polymer chains/fibers;
providing electrically conductive nanodots and functionalizing the electrically conductive nanodots with preselected functional groups R′ to an outer surface of the electrically conductive nanodots to producing functionalized electrically conductive nanodots; and
mixing the functionalized electrically conductive nanodots and the functionalized polymer chains/fibers with crosslinker molecules having opposed ends under conditions suitable induce formation of cross linkages between the electrically conductive nanodots and polymeric matrix through the crosslinker molecules having one end thereof bound to the R″ functional groups on the polymer strands and the other end of the crosslinker molecules bound to the R′ functional groups on the electrically conductive nanodots.

12. The method according to claim 11, wherein the physiologically acceptable polymer is any one or combination of synthetic polymers and natural polymers.

13. The method according to claim 12, wherein the natural polymers include any one or combination of collagen, agarose, gelatine, fibrin, elastin cellulose, silk fibroin, chitin, chitosan, glycosaminoglycans, keratin, pectin, hyaluronic acid, lactate, starch and lignin.

14. The method according to claim 12, wherein the synthetic polymers include any one or combination of Polyethylene (PE), Polypropylene (PP), Polycarbonate (PC), Polyimide (PI), Polystyrene (PS), Poly(lactic-co-glycolic acid) (PLGA), Polylactic acid (PLA), Poly(lactic acid)-graft-poly(methacrylic acid) (PLA-g-P(MAA)), Poly-L-lysine (PLL), Polyethylene terephthalate (PET), Gelatin methacryloyl (GelMA), Polyethylene glycol diacrylate (PEGDA), Polyethylene glycol (PEG), Polyethylene oxide (PEO), Polyvinyl chloride (PVC), Polymethyl methacrylate (PMMA), Polytetrafluoroethylene (PTFE), Polycaprolactone (PCL), Polyvinyl alcohol (PVA), Polydimethylsiloxane (PDMS), Polyether ether ketone (PEEK), Polyhydroxyalkanoate (PHA).

15. The method according to claim 11, wherein the functional groups R″ are any one or combination of NH₂, NH, NCO, CH₃, CH₂, COOH, CO, OH, SH, SO₃, H and O, and wherein the functional groups R′ are any one or combination of NH₂, COOH, NH, OH, SH, G, SiOH, PO₃H₂, SO₃— and CONH₂.

16. The method according to claim 11, wherein the electrically conductive nanodots are any one or combination of carbon nanodots, graphene nanodots, metallic nanodots, semiconductor nanodots, silicon nanodots, indium tin oxide nanodots, copper nanodots and zinc oxide nanodots.

17. The method according to claim 11, wherein the electrically conductive nanodots have a diameter in a range from about 1 nm to about 100 nanometers.

18. The method according to claim 11, wherein the crosslinker molecules are bound to the functional groups R′ and R″ by any one or combination of covalent bonding, electrostatic and cathecol-based interactions.

19. The method according to claim 11, wherein the crosslinker agents are any one or combination of molecules capable of creating permanent covalent chemistries (amine, amide, urea, thioether, siloxane, acrylamide bonds) and dynamic covalent chemistries (imine, acylhydrazone, oxime,boronic-ester, disulfide, thioester, bonds), ions capable of creating electrostatic interactions (cation-anion, bridging ion, cation-π, anion-π, π-π interactions, hydrogen bonding), and molecules for other classes of interactions (cyclodextrin-based, cathecol-based, cucurbit[n]uril-based).
